(19) United States
(12) Patent Application Publication
(10) Pub. No.: US 2007/0142859 A1
(43) Pub. Date: Jun. 21, 2007

(54) EMBOLIC COILS

(75) Inventors: Marcia S. Buiser, Watertown, MA (US); Ashley Seehusen, Newton, MA (US); Christopher Nardone, North Chelmsford, MA (US); Paul D. DiCarlo, Middleboro, MA (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/458,156

(22) Filed: Jul. 18, 2006

Related U.S. Application Data
(63) Continuation-in-part of application No. 11/311,617, filed on Dec. 19, 2005.
(63) Continuation-in-part of application No. 11/430,602, filed on May 9, 2006.

(51) Int. Cl. A61M 29/00

(52) U.S. Cl. 606/200

(57) ABSTRACT

Coils, such as embolic coils, and related methods, devices, and compositions, are disclosed.

### CROSS-REFERENCE TO RELATED APPLICATIONS

This application is a continuation-in-part of, and claims priority under 35 U.S.C. §120 to, U.S. patent application Ser. No. 11/311,617, filed on Dec. 19, 2005, and U.S. patent application Ser. No. 11/430,602, filed on May 9, 2006, both of which are incorporated herein by reference in their entirety.

### TECHNICAL FIELD

The invention relates to coils, such as embolic coils, as well as related methods, devices, and compositions.

### BACKGROUND

Therapeutic vascular occlusions (embolizations) are used to prevent or treat pathological conditions in situ. Embolic coils can be used to occlude vessels in a variety of medical applications. Delivery of embolic coils (e.g., through a catheter) can depend on the size and/or shape of the coils. Some embolic coils include fibers that can, for example, enhance thrombosis at a treatment site.

### SUMMARY

In one aspect, the invention features an article including an embolic coil body, at least one fiber (e.g., a plurality of fibers) attached to the embolic coil body, and a material supported by the embolic coil body and/or the fiber.

In another aspect, the invention features an article including an embolic coil body, a plurality of fibers attached to the embolic coil body, and a coating including a gel. The coating contacts the embolic coil body and the fibers.

In an additional aspect, the invention features a medical device including a tubular body defining a lumen, and at least one article (e.g., a plurality of articles) disposed within the lumen. The article includes an embolic coil body, at least one fiber (e.g., a plurality of fibers) attached to the embolic coil body, and a material supported by the embolic coil body and/or the fiber.

In a further aspect, the invention features a method including administering an article to a subject. The article includes an embolic coil body, at least one fiber (e.g., a plurality of fibers) attached to the embolic coil body, and a material supported by the embolic coil body and/or the fiber.

In an additional aspect, the invention features a method of coating an article. The article includes an embolic coil body and at least one fiber (e.g., a plurality of fibers) attached to the embolic coil body. The method includes contacting the embolic coil body and/or the fiber with a material, and forming a coating including the material. The coating is supported by the embolic coil body and/or the fiber.

In a further aspect, the invention features a method including administering a medical device to a subject. The medical device includes a tubular body defining a lumen, and at least one article (e.g., a plurality of articles) disposed within the lumen. The article includes an embolic coil body, at least one fiber (e.g., a plurality of fibers) attached to the embolic coil body, and a material supported by the embolic coil body and/or the fiber.

Embodiments can also include one or more of the following.

The material can be bioerodible and/or bioabsorbable. The material can include a gel and/or a polymer. In some embodiments, the material can include one or more of the following: polysaccharides; polysaccharide derivatives; inorganic, ionic salts; water soluble polymers; biodegradable poly DL-lactide-poly ethylene glycol (PELA); hydrogels; polyethylene glycol (PEG); chitosan; polyesters; poly(lactic-co-glycolic) acid; polyamino acids; polynucleic acids; polyhydroxyalkanoates; polyanhydrides; polylactic acids (PLA); alginate salts (e.g., sodium alginate); carboxymethyl cellulose; ethylenediaminetetraacetic acid (EDTA); polyvinyl alcohols (PVA); polyacrylic acids; polymethacrylic acids; poly vinyl sulfonates; carboxymethyl celluloses; hydroxyethyl celluloses; substituted celluloses; polyacrylamides; polyethylene glycols; polyamides (e.g., nylon); polyureas; polyurethanes; polyesters; polyethers; polystyrenes; polysaccharides; polylactic acids; polyethylenes; polymethylmethacrylates; polyethylacrylate; polycaprolactones; polyglycolic acids (PGA); poly(lactic-co-glycolic) acids; polyvinylpyrrolidone; methacrylates; cellulose esters; carbohydrates. In some embodiments, the material can include one or more block copolymers, such as styrene-isobutylene-styrene (SIBS) and/or styrene-ethylene/butylene-styrene (SEBS).

The material can be in the form of a coating on the embolic coil body and/or the fiber. In some embodiments, the coating can have a thickness of at least 0.0001 inch (e.g., at least 0.001 inch, at least 0.002 inch, at least 0.005 inch) and/or at most 0.02 inch (e.g., at most 0.005 inch, at most 0.002 inch, at most 0.001 inch).

In certain embodiments, the material may not be supported by the embolic coil body. In some embodiments, the material may not be supported by the fiber.

The embolic coil body can include a plurality of windings of at least one wire. The wire can include a metal (e.g., platinum) and/or a metal alloy (e.g., stainless steel). In certain embodiments, the material can be supported by the wire.

The fiber can include a polyamide and/or a polyester. The material can be supported by the fiber (e.g., the material can be in the form of a coating on the fiber). In some embodiments in which the article includes a plurality of fibers, the fibers can be in the form of a fiber bundle. In certain embodiments, the material can be supported by the fiber bundle (e.g., the material can be in the form of a coating on the fiber bundle).

The article can include a therapeutic agent, such as heparin. In some embodiments, the therapeutic agent can be dispersed within the material.

In certain embodiments, the article can include at least two (e.g., three, four, five, 10) materials. In some embodiments, the materials can be combined with each other (e.g., in a mixture). For example, in some embodiments, one material can be dispersed within another material. In certain embodiments, at least one of the materials can be a polymer. In some embodiments, at least one of the materials can be a nitric oxide donor. In certain embodiments, the article can include at least two materials that are bioerodible and/or bioabsorbable.

The embolic coil body can have a primary coil shape having a length of at least about 0.2 centimeter (e.g., at least about two centimeters, at least about 30 centimeters) and/or at most about 100 centimeters (e.g., at most about 30 centimeters, at most about two centimeters).

In some embodiments, the tubular body can be a catheter. In certain embodiments, the tubular body can be an introducer sheath.

The method can include contacting the embolic coil body, the fiber, or both the embolic coil body and the fiber, with the material. In some embodiments, the embolic coil body can include a plurality of windings of at least one wire, and contacting the embolic coil body with the material can include contacting the wire with the material. In certain embodiments, the method may not include contacting the fiber with the material. In some embodiments, the method may not include contacting the embolic coil body with the material.

Embodiments can include one or more of the following advantages.

In some embodiments, a coil can exhibit relatively good occlusive properties when delivered to a target site within a subject. For example, in certain embodiments, a coil can include fibers that can enhance thrombosis at the target site, thereby enhancing occlusion of the target site. A coil with relatively good occlusive properties can be used, for example, to occlude a vessel (e.g., to embolize a tumor), treat an aneurysm, treat an arteriovenous malformation, and/or treat a fistula (e.g., a traumatic fistula).

In certain embodiments, a coil (e.g., a coil that includes a coating) can have a relatively low likelihood of sticking to a wall of a delivery device (e.g., a catheter, an introducer sheath). This can, for example, reduce the possibility of complications resulting from the coil sticking to a wall of the delivery device when the coil is being delivered to a target site within a subject.

In some embodiments, a coil (e.g., a coil that includes a coating) can exhibit relatively good deliverability. For example, in certain embodiments, a coil that includes fibers coated by a bioerodible and/or bioabsorbable material can experience relatively little friction with the walls of a delivery device if the coil contacts the walls of the delivery device during delivery. The coating on the fibers can enhance the lubricity of the coil, making it relatively easy to deliver the coil from a delivery device.

In some embodiments, a coil (e.g., a coil that includes a coating) can have a relatively smooth outer surface. The relatively smooth outer surface may enhance the deliverability of the coil from a delivery device (e.g., by limiting the likelihood that the coil will become caught on the delivery device during delivery).

In certain embodiments, a coil that includes fibers and a coating can have a relatively high effective column strength. The coating can increase the effective column strength of the coil by, for example, aligning and/or orienting the fibers so that they are relatively close to the coil body. In some embodiments, a coil with a relatively high effective column strength can be relatively easy to deliver from a delivery device, such as a catheter. For example, even if the coil sticks to the walls of the delivery device, the effective column strength of the coil can be sufficiently high to allow an operator of the delivery device to overcome the sticking and deploy the coil from the delivery device.

In certain embodiments, a coil that includes fibers and a coating can have a relatively low profile. For example, the coating can align and/or orient the fibers so that the fibers do not protrude substantially from the coil body. A coil with a relatively low profile can, for example, be relatively easy to deliver to a target site.

In some embodiments, a fibered coil can be relatively unlikely to lose its fiber(s) during delivery. For example, in certain embodiments, the fibers of a fibered coil can be protected during delivery by a coating.

In certain embodiments, a coil can be used to deliver one or more therapeutic agents to a target site. In some embodiments, a coil can be used to deliver a metered dose of a therapeutic agent to a target site over a period of time. In certain embodiments, the release of a therapeutic agent from a coil can be delayed until the coil has reached a target site. For example, in some embodiments, a coil can include a bioerodible coating that erodes during delivery, such that when the coil reaches the target site, the coil can begin to release the therapeutic agent.

In some embodiments, a coil can be used to deliver multiple therapeutic agents, either to the same target site, or to different target sites. For example, a coil can deliver one type of therapeutic agent (e.g., an anti-inflammatory agent, an anti-thrombotic agent) as the coil is being delivered to a target site, and another type of therapeutic agent (e.g., a growth factor) once the coil has reached the target site.

Other aspects, features, and advantages are in the description, drawings, and claims.

### DESCRIPTION OF DRAWINGS

FIG. 1A is a side view of an embodiment of an embolic coil.

FIG. 1B is a cross-sectional view of the embolic coil of FIG. 1A, taken along line 1B-1B.

FIG. 1C is an enlarged view of region 1C of the embolic coil of FIG. 1B.

FIG. 1D is a cross-sectional view of the embolic coil of FIG. 1B, taken along line 1D-1D.

FIGS. 2A-2E illustrate the delivery of the embolic coil of FIGS. 1A-1D to the site of an aneurysm.

FIG. 3 is a perspective view of an embodiment of an embolic coil.

FIG. 4 is a perspective view of an embodiment of an embolic coil.

FIG. 5 is a perspective view of an embodiment of an embolic coil.

FIG. 6 is a perspective view of an embodiment of an embolic coil.

FIG. 7A is a front view of an embodiment of an embolic coil.

FIG. 7B is a side view of the embolic coil of FIG. 7A.

FIG. 8A is a front view of an embodiment of an embolic coil.

FIG. 8B is a side view of the embolic coil of FIG. 8A.

FIG. 9 is a side view of an embodiment of an embolic coil.

FIG. 10A illustrates an embodiment of a process for forming an embolic coil.

FIG. 10B is a side view of an embodiment of a mandrel used in the process of FIG. 10A.

FIG. 10C is a cross-sectional view of the mandrel of FIG. 10B, taken along line 10C-10C.

FIG. 11A is a side view of an embodiment of a mandrel.

FIGS. 11B and 11C illustrate an embodiment of a process for forming an embolic coil using the mandrel of FIG. 11A.

FIG. 12A illustrates an embodiment of a process for forming an embolic coil.

FIG. 12B is a perspective view of an embodiment of an embolic coil formed using the process of FIG. 12A.

FIGS. 13A-13D illustrate an embodiment of a process for forming an embolic coil.

FIG. 13E is a perspective view of an embodiment of an embolic coil formed using the process of FIGS. 13A-13D.

FIG. 14 is a side view in partial cross-section of an embodiment of an embolic coil.

FIG. 15 is a side view in partial cross-section of an embodiment of an embolic coil.

FIG. 16 is a side view in partial cross-section of an embodiment of an embolic coil.

FIG. 17 is a side view in partial cross-section of an embodiment of an embolic coil.

FIG. 18A is a side view in partial cross-section of an embodiment of an embolic coil.

FIG. 18B is a cross-sectional view of the embolic coil of FIG. 18A, taken along line 18B-18B.

FIG. 19 illustrates the delivery of an embodiment of an embolic coil from an introducer sheath into a delivery device.

FIG. 20A is a side view of an embodiment of an apparatus for coating an embolic coil.

FIG. 20B is a cross-sectional view of the apparatus of FIG. 20A, taken along line 20B-20B.

FIG. 20C illustrates, in partial cross-section, the use of the apparatus of FIGS. 20A and 20B to coat an embodiment of an embolic coil.

FIG. 21 is a side view of an embodiment of an embolic coil.

### DETAILED DESCRIPTION

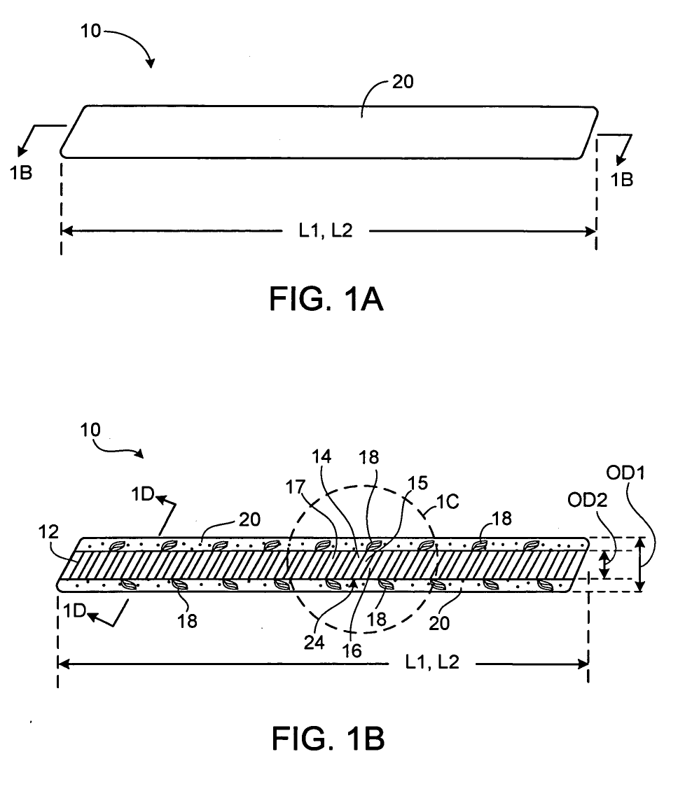

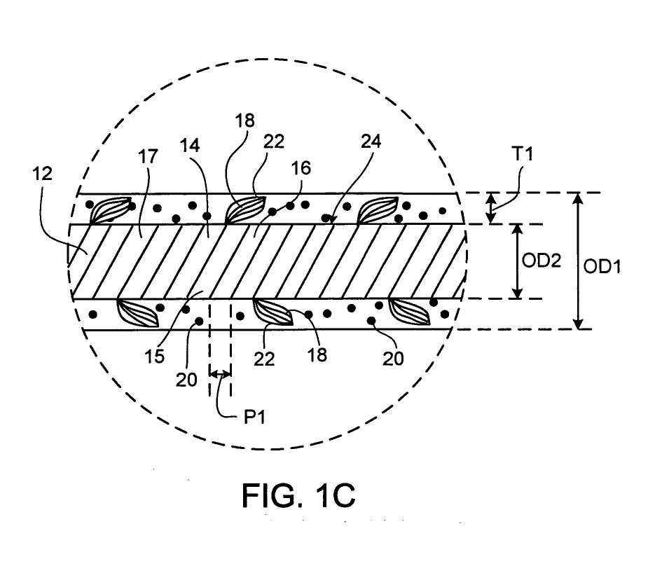

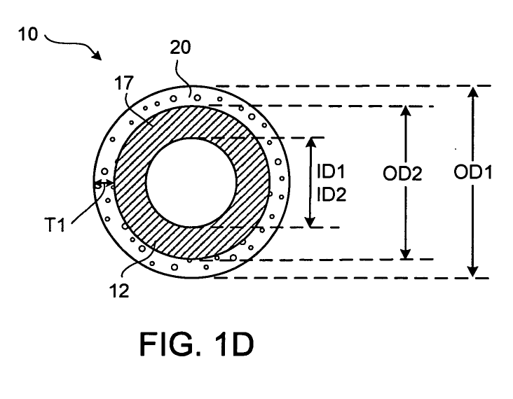

FIGS. 1A-1D show the primary shape of an embolic coil 10 that includes an embolic coil body 12, fiber bundles 18 attached to coil body 12, and a coating 20. Coil body 12 is formed of windings (e.g., windings 14, 15, and 16) of a wire 17 (e.g., a platinum wire). Fiber bundles 18 are formed of fibers 22 (e.g., polyester fibers). Coating 20 is disposed on the exterior surface 24 of coil body 12, and encapsulates fiber bundles 18. Embolic coil 10 may be used, for example, in an embolization procedure, and/or may be used to deliver one or more therapeutic agents to a target site. Coating 20 can enhance the deliverability of embolic coil 10 by, for example, limiting the likelihood of fibers 22 coming into contact with, and/or sticking to, the walls of a delivery device. In some embodiments, during and/or after delivery of embolic coil 10 to a target site, coating 20 can erode and/or can be absorbed, which can allow fiber bundles 18 to become exposed. Fiber bundles 18 can then be used to enhance the treatment of the target site (e.g., by enhancing occlusion of the target site).

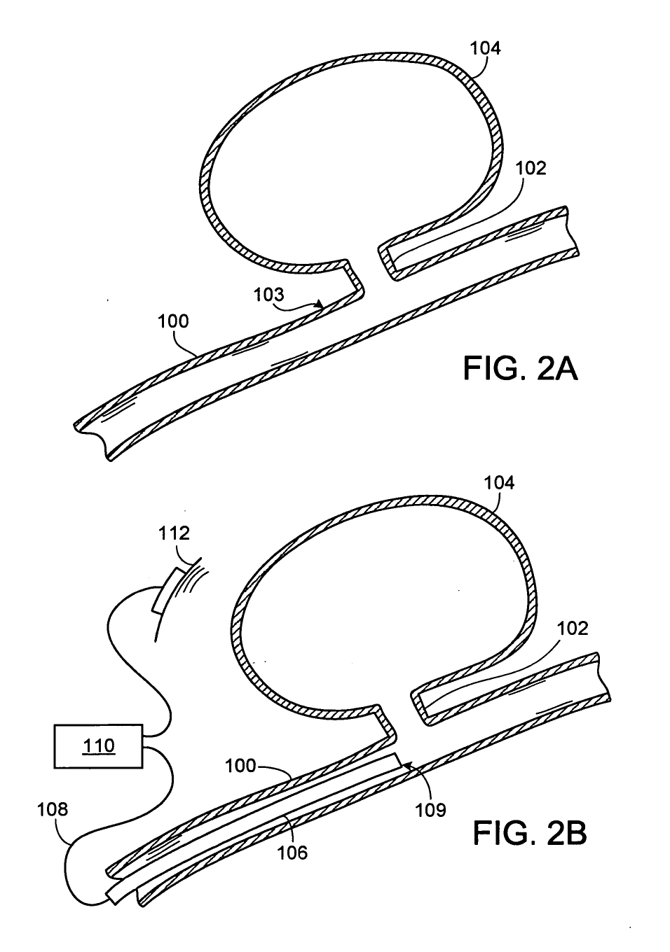

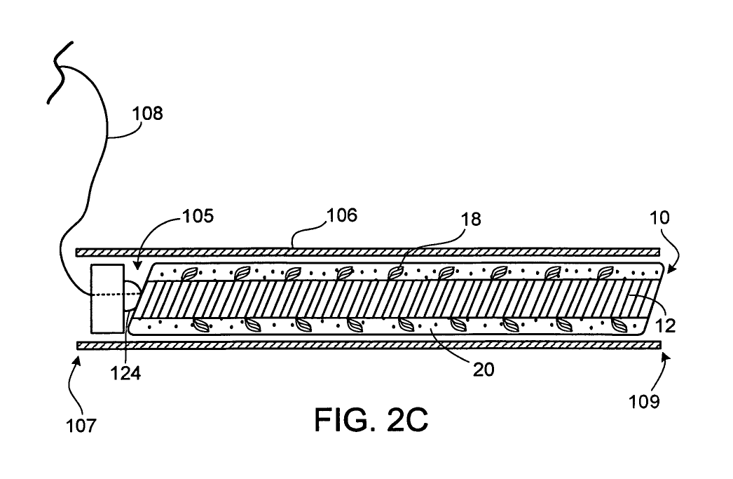

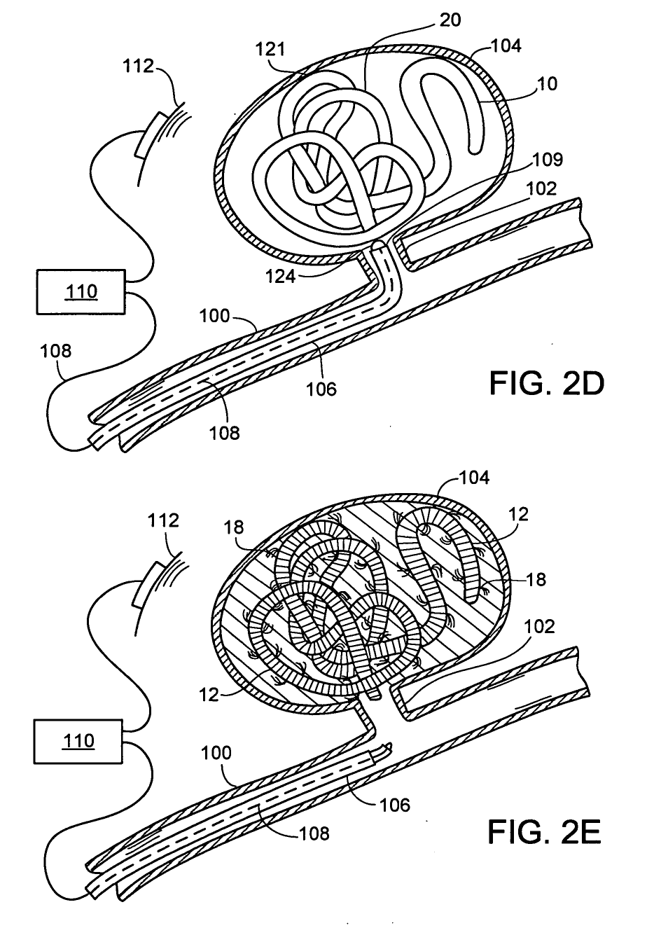

FIGS. 2A-2E show the use of embolic coil 10 to fill and occlude an aneurysmal sac 104 of a subject. As shown in FIG. 2A, aneurysmal sac 104 is formed in a wall 103 of a vessel 100, and is connected to vessel 100 by a neck 102.

As FIG. 2B shows, a catheter 106 containing embolic coil 10 is delivered into vessel 100. FIG. 2C shows a cross-sectional view of catheter 106 containing embolic coil 10. As shown in FIG. 2C, catheter 106 has a proximal end 107 and a distal end 109. Embolic coil 10 is disposed within a lumen 105 of catheter 106, and is in its primary shape. In some embodiments, embolic coil 10 can be disposed within a pharmaceutically acceptable carrier (e.g., a saline solution, a contrast agent, a heparin solution, a heparinized saline solution) while embolic coil 10 is within lumen 105 of catheter 106. In certain embodiments, embolic coil 10 may not be disposed in any carriers while embolic coil 10 is within lumen 105 of catheter 106. Catheter 106 includes a core wire 108 connected to a power supply 110. Power supply 110 has a negative pole 112 that can be placed in electrical contact with the skin of the subject. Alternatively or additionally, mechanical detachment mechanisms (e.g., an interlocking detachable coil mechanism) may be used.

As shown in FIG. 2D, catheter 106 is used to deliver embolic coil 10 into aneurysmal sac 104, at least until a sacrificial link 124 between embolic coil 10 and core wire 108 is exposed beyond the distal end 109 of catheter 106. When an electrical current generated by power supply 110 flows through core wire 108, the electrical current causes sacrificial link 124 to disintegrate, thereby electrolytically detaching embolic coil 10 from core wire 108. As shown in FIG. 2E, embolic coil 10 fills aneurysmal sac 104, helping to occlude aneurysmal sac 104.

During and/or after delivery of embolic coil 10 into aneurysmal sac 104, coating 20 erodes and/or is absorbed, eventually resulting in the exposure of fiber bundles 18 (FIG. 2E). Fiber bundles 18 can accelerate the occlusion of aneurysmal sac 104 by, for example, enhancing thrombosis within aneurysmal sac 104. An accelerated embolization procedure can benefit the subject (e.g., by reducing exposure time to fluoroscopy). Embolic coils and coil delivery are described, for example, in Elliott et al., U.S. Patent Application Publication No. US 2006/0116711 A1, published on Jun. 1, 2006, and entitled “Embolic Coils”; Buiser et al., U.S. patent application Ser. No. 11/430,602, filed on May 9, 2006, and entitled “Embolic Coils”; and Buiser et al., U.S. patent application Ser. No. 11/311,617, filed on Dec. 19, 2005, and entitled “Coils”, all of which are incorporated herein by reference.

The presence of a coating such as coating 20 on a fibered embolic coil can enhance the deliverability (e.g., by increasing the pushability) of the embolic coil.

As an example, in some embodiments, the coating can help to limit movement by the fibers on the coil while the coil is being loaded into a delivery device, an/or while the coil is being delivered from a delivery device. This can, for example, reduce the likelihood of the fibers sticking to the delivery device and slowing the loading and/or delivery process.

As another example, in certain embodiments, a fibered embolic coil that includes a coating can have a relatively high effective column strength. The effective column strength of embolic coil 10 is the column strength (the compression load at which embolic coil 10 will buckle) of embolic coil 10 when the embolic coil is constrained within lumen 105 of catheter 106. The presence of coating 20 on embolic coil 10 can cause embolic coil 10 to have a relatively high effective column strength. Because of its relatively high effective column strength, embolic coil 10 can also have good pushability. Thus, even if fibers 22 stick to catheter 106 during delivery of embolic coil 10, embolic coil 10 can have sufficiently good pushability to overcome the sticking, thereby allowing embolic coil 10 to be deployed from catheter 106 relatively easily. In some embodiments, an embolic coil with relatively good pushability can be less likely to buckle during deployment from a delivery device than an otherwise comparable embolic coil with relatively low pushability.

As an additional example, in some embodiments, a fibered embolic coil that includes a coating can be loaded into a delivery device (e.g., a catheter) starting at either the proximal end of the delivery device or the distal end of the delivery device. For example, the coil can be loaded into the proximal end of a catheter, and then can be pushed toward the distal end of the catheter.

While the treatment of an aneurysmal sac using embolic coil 10 has been described, embolic coils such as embolic coil 10 can generally be used in a number of different applications, such as neurological applications and/or peripheral applications. In some embodiments, embolic coils can be used to embolize a lumen of a subject (e.g., to occlude a vessel), and/or to treat an aneurysm (e.g., an intercranial aneurysm), an arteriovenous malformation (AVM), and/or a fistula (e.g., a traumatic fistula). In certain embodiments, embolic coils can be used to embolize a tumor (e.g., a liver tumor), and/or to control tumor growth. In some embodiments, embolic coils can be used in transarterial chemoembolization (TACE). In certain embodiments, embolic coils can be used to obstruct blood flow in a region of a subject prior to surgical resection and/or radiosurgery.

As described above, the erosion and/or absorption of coating 20 can result in the exposure of fiber bundles 18. Coating 20 can include (e.g., can be formed of) one or more materials. Typically, coating 20 can include at least one bioerodible and/or bioabsorbable material (e.g., a polymer). In some embodiments, the bioerodible and/or bioabsorbable material can begin to erode and/or to be absorbed upon contact with blood and/or other body fluids. In certain embodiments, coating 20 can include at least two different bioerodible and/or bioabsorbable materials that are combined with each other (e.g., in a mixture). In some embodiments, coating 20 can be formed entirely of at least one bioerodible and/or bioabsorbable material.

Examples of bioerodible and/or bioabsorbable materials include polysaccharides (e.g., alginate, agarose); polysaccharide derivatives; inorganic, ionic salts; water soluble polymers (e.g., polyvinyl alcohol, such as polyvinyl alcohol that has not been cross-linked); biodegradable poly DL-lactide-poly ethylene glycol (PELA); hydrogels (e.g., polyacrylic acid, hyaluronic acid, gelatin, carboxymethyl cellulose); polyethylene glycol (PEG); chitosan; polyesters (e.g., polycaprolactones); poly(lactic-co-glycolic) acid (e.g., a poly(d-lactic-co-glycolic) acid); polyamino acids; polynucleic acids; polyhydroxyalkanoates; polyanhydrides; polylactic acids; alginate salts (e.g., sodium alginate); and combinations thereof. In some embodiments, a bioerodible and/or bioabsorbable material can include carboxymethyl cellulose, sodium alginate, ethylenediaminetetraacetic acid (EDTA), or a combination thereof.

Coating 20 can include other materials. For example, in some embodiments, coating 20 can include one or more of the following polymers: polyvinyl alcohols (PVA), polyacrylic acids, polymethacrylic acids, poly vinyl sulfonates, carboxymethyl celluloses, hydroxyethyl celluloses, substituted celluloses, polyacrylamides, polyethylene glycols, polyamides (e.g., nylon), polyureas, polyurethanes, polyesters, polyethers, polystyrenes, polysaccharides, polylactic acids, polyethylenes, polymethylmethacrylates, polyethylacrylate, polycaprolactones, polyglycolic acids, poly(lactic-co-glycolic) acids (e.g., poly(d-lactic-co-glycolic) acids), polyvinylpyrrolidone; and copolymers or mixtures thereof. An example of a copolymer is a polyglycolic acid/lactide copolymer. Other examples of copolymers include styrene-isobutylene-styrene (SIBS) and styrene-ethylene/butylene-styrene (SEBS). In certain embodiments, coating 20 can include a highly water insoluble, high molecular weight polymer. An example of such a polymer is a high molecular weight polyvinyl alcohol (PVA) that has been acetalized. The polymer can be substantially pure intrachain 1,3-acetalized PVA and substantially free of animal derived residue such as collagen.

In some embodiments, coating 20 can include one or more methacrylates, cellulose esters, and/or carbohydrates.

In certain embodiments, coating 20 can include one or more other materials, such as the Medi-Coat™ hemocompatible coating from Angiotech BioCoatings Corp. (Henrietta, N.Y.). Medi-Coat™ hemocompatible coating is formed of heparin entrapped in hybrid polymer layers (e.g., cellulose esters, polyurethanes, methacrylates, polyvinylpyrrolidone). Another example of a material is Carmeda® Bioactive Surface (CBAS™), from Carmeda Inc. (San Antonio, Tex.). Heparin, which can limit or prevent thrombosis, can be attached to the end points of the Carmeda® Bioactive Surface, so that interaction between the heparin and flowing blood can be maximized, and thrombosis can be minimized.

In some embodiments, coating 20 can include one or more gelling precursors. Examples of gelling precursors include alginates, alginate salts (e.g. sodium alginate), xanthan gums, natural gum, agar, agarose, chitosan, carrageenan, fucoidan, furcellaran, laminaran, hypnea, eucheuma, gum arabic, gum ghatti, gum karaya, gum tragacanth, hyaluronic acid, locust beam gum, arabinogalactan, pectin, amylopectin, other water soluble polysaccharides and other ionically cross-linkable polymers. A particular gelling precursor is sodium alginate. An example of sodium alginate is high guluronic acid, stem-derived alginate (e.g., about 50 percent or more, about 60 percent or more guluronic acid) with a low viscosity (e.g., from about 20 centipoise to about 80 centipoise at 20° C.). As used herein, the viscosity of alginate is measured using a digital cone/plate viscometer from Brookfield Engineering at a temperature of from 65° C. to 75° C. and a spindle speed of from 1.5 rpm to 3.0 rpm.

In certain embodiments, coating 20 can include one or more proteins. Examples of proteins include collagen, enzymes, and growth factors.

In some embodiments, coating 20 can include one or more gelled materials, and/or can be in a gel form. As an example, in certain embodiments, coating 20 can be formed of a gelling precursor (e.g., alginate) that has been gelled by being contacted with a gelling agent (e.g., calcium chloride). As another example, in some embodiments, coating 20 can be formed of a saline gel, such as Normlgel® 0.9% Isotonic Saline Gel (from Mölynlycke Health Care, Göteborg, Sweden). Saline gels can be relatively inert (e.g., unlikely to have an adverse effect on the body of a subject), and/or can be water-soluble.

In certain embodiments, coating 20 can include one or more materials that are nitric oxide donors. Without wishing to be bound by theory, it is believed that endothelial cells can generate nitric oxide (NO), which can limit or prevent platelet activation and/or thrombosis. It is further believed that a coating including a nitric oxide donor can mimic this effect. Examples of nitric oxide donors include nitrosothiols; organic nitrates/nitrites (e.g., nitroglycerin, isosorbide dinitrate, amyl nitrite); inorganic nitroso compounds (e.g., sodium nitroprusside); sydnonimines (e.g., molsidomine, linsidomine); nonoates (e.g., diazenium diolates, NO adducts of alkanediamines); S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers); C-nitroso-, O-nitroso- and N-nitroso-compounds; and L-arginine. In some embodiments, coating 20 can include one or more polymers (e.g., polyvinyl chloride) and one or more nitric oxide donors. For example, coating 20 can include a polymer entrapping a nitric oxide donor. Polymers entrapping nitric oxide donors are available, for example, from MC3, Inc. (Ann Arbor, Mich.).

Typically, the concentration of nitric oxide donors in coating 20 can be selected to limit and/or prevent clotting during delivery of embolic coil 10, but to allow clotting once embolic coil 10 has been delivered to a target site. In certain embodiments, coating 20 can include at least about five percent by weight (e.g., at least about 10 percent by weight, at least about 20 percent by weight, at least about 30 percent by weight), and/or at most about 40 percent by weight (e.g., at most about 30 percent by weight, at most about 20 percent by weight, at most about 10 percent by weight), nitric oxide donors.

In some embodiments, coating 20 can include one or more materials that can camouflage coil body 12 and/or fiber bundles 18 (e.g., limiting the body's ability to recognize embolic coil 10 as a foreign object). This can, for example, result in a reduced likelihood of embolic coil 10 eliciting a response from the body, such as clot formation. An example of a material that can be used to camouflage coil body 12 and/or fiber bundles 18 is the Camouflage® glycocompound coating, from Hemoteq GmbH (Wuerselen, Germany). The Camouflage® glycocompound coating includes synthetic carbohydrates that can mimic endothelial cells in human blood vessels. Another example of a camouflaging material is a material that can attract and bind blood proteins to the surface of an embolic coil that is coated with the material. The proteins can eventually cover the coated surface, causing the coated surface to mimic endothelial cells, and thereby limiting or preventing clot formation.

In certain embodiments, an embolic coil can include both a camouflaging coating and a bioerodible and/or bioabsorbable material. For example, an embolic coil can include a coil body that is coated with a camouflaging coating, and can include fiber bundles that are coated with a bioerodible and/or bioabsorbable material.

In certain embodiments, coating 20 can include one or more radiopaque materials. As used herein, a radiopaque material refers to a material having a density of about ten grams per cubic centimeter or greater (e.g., about 25 grams per cubic centimeter or greater, about 50 grams per cubic centimeter or greater). In some embodiments in which coating 20 includes one or more radiopaque materials, embolic coil 10 can exhibit enhanced visibility under X-ray fluoroscopy, such as when embolic coil 10 is in a subject. In certain embodiments, X-ray fluoroscopy can be performed without the use of a radiopaque contrast agent. Radiopaque materials are described, for example, in Rioux et al., U.S. Patent Application Publication No. US 2004/0101564, published on May 27, 2004, and entitled “Embolization”, which is incorporated herein by reference.

In some embodiments, coating 20 can include one or more MRI-visible materials. As used herein, an MRI-visible material refers to a material that has a magnetic susceptibility of at most about one or less (e.g., at most about 0.5 or less; at most about zero or less) when measured at 25° C. In some embodiments in which coating 20 includes one or more MRI-visible materials, embolic coil 10 can exhibit enhanced visibility under MRI, such as when embolic coil 10 is in a subject (see discussion below). In certain embodiments, MRI can be performed without the use of an MRI contrast agent. Examples of MRI-visible materials include superparamagnetic iron oxides (SPIO). MRI-visible materials are described, for example, in Rioux et al., U.S. Patent Application Publication No. US 2004/0101564, published on May 27, 2004, and entitled “Embolization”, which is incorporated herein by reference.

In certain embodiments, coating 20 can include one or more ferromagnetic materials. As used herein, a ferromagnetic material refers to a material that has a magnetic susceptibility of at least about 0.075 or more (e.g., at least about 0.1 or more; at least about 0.2 or more; at least about 0.3 or more; at least about 0.4 or more; at least about 0.5 or more; at least about one or more; at least about ten or more; at least about 100 or more; at least about 1,000 or more; at least about 10,000 or more) when measured at 25° C. In some embodiments in which coating 20 includes one or more ferromagnetic materials, a magnetic source can be used to move or direct embolic coil 10 to a treatment site. The magnetic source can be external to the subject's body, or can be used internally. In certain embodiments, both an external magnetic source and an internal magnetic source can be used to move embolic coil 10. An example of an internal magnetic source is a magnetic catheter. Magnetic catheters are described, for example, in Freyman, U.S. Patent Application Publication No. US 2003/0187320 A1, published on Oct. 2, 2003, and entitled “Magnetically Enhanced Injection Catheter”, which is incorporated herein by reference. An example of an external magnetic source is a magnetic wand. Ferromagnetic materials are described, for example, in Rioux et al., U.S. Patent Application Publication No. US 2004/0101564, published on May 27, 2004, and entitled “Embolization”, which is incorporated herein by reference.

In some embodiments, coating 20 can include one or more materials that are neither bioerodible nor bioabsorbable.

In certain embodiments, coating 20 can include two or more of any of the above materials.

In general, fibers 22 can include (e.g., can be formed of) one or more materials that can enhance thrombosis (e.g., at a target site). In some embodiments, fibers 22 can include one or more polyesters, such as polyethylene terephthalate (e.g., Dacron®). In certain embodiments, fibers 22 can include one or more polyamides (e.g., nylon), and/or can include collagen. Fibers 22 can have a length of at least about 0.5 millimeter (e.g., at least about one millimeter, at least about five millimeters) and/or at most about 10 millimeters (e.g., at most about five millimeters, at most about one millimeter). In some embodiments, the length of fibers 22 can be selected so that fibers 22 can be fully coated by coating 20.

Fibers on an embolic coil, such as fibers 22, can, for example, be snapped between one or more windings of the embolic coil body, and/or can be bonded the coil body (e.g., by an adhesive). While fibers 22 are shown in the form of fiber bundles 18, in some embodiments, an embolic coil can include fibers that are not in the form of fiber bundles.

In certain embodiments, an embolic coil can include at least one fiber that is a suture. Examples of sutures include bioabsorbable sutures (e.g., polyglycolide sutures), non-bioabsorbable sutures (e.g., expandable polytetrafluoroethylene sutures, polyethylene terephthalate sutures), synthetic sutures (e.g., polypropylene sutures, nylon sutures), and natural sutures (e.g., catgut sutures, collagen sutures).

Fibers are described, for example, in Elliott et al., U.S. Patent Application Publication No. US 2006/0116711 A1, published on Jun. 1, 2006, and entitled “Embolic Coils”, which is incorporated herein by reference.

In general, wire 17 can include (e.g., can be formed of) one or more materials (e.g., biocompatible materials) that allow wire 17 to be wound into a coil shape. Wire 17 can include, for example, one or more metals or metal alloys, such as platinum, platinum alloys (e.g., platinum-tungsten alloys), stainless steel, nitinol, and/or Elgiloy® (from Elgiloy Specialty Metals).

As shown in FIGS. 1A-1D, embolic coil 10 in its primary shape has a length L1, an inner diameter ID1, an outer diameter OD1 and a thickness T1. In some embodiments, length L1 can be at least about 0.2 centimeter (e.g., at least about two centimeters, at least about 2.3 centimeters, at least about 30 centimeters, at least about 50 centimeters, at least about 80 centimeters) and/or at most about 100 centimeters (e.g., at most about 80 centimeters, at most about 50 centimeters, at most about 30 centimeters, at most about 2.3 centimeters, at most about two centimeters). In certain embodiments, length L1 can be from about 2.3 centimeters to about 30 centimeters. In some embodiments, inner diameter ID1 can be at least 0.0005 inch (e.g., at least 0.01 inch, at least 0.015 inch, at least 0.02 inch) and/or at most 0.0698 inch (e.g., at most 0.5 inch, at most 0.3 inch, at most 0.015 inch, at most 0.01 inch, at most 0.005 inch). In some embodiments, outer diameter OD1 can be at least 0.0027 inch (e.g., at least about 0.005 inch, at least 0.01 inch, at least 0.016 inch, at least 0.02 inch, at least 0.03 inch) and/or at most 0.072 inch (e.g., at most about 0.06 inch, at most about 0.05 inch, at most about 0.04 inch, at most 0.03 inch, at most 0.02 inch, at most 0.016 inch, at most 0.01 inch).

In certain embodiments, outer diameter OD1 can be selected based on the application of embolic coil 10. As an example, in some embodiments in which embolic coil 10 can be used to treat intracranial aneurysms, outer diameter OD1 can be relatively small (e.g., at most 0.016 inch). As another example, in certain embodiments in which embolic coil 10 can be used to treat arteriovenous malformations (AVM), outer diameter OD1 can be relatively large (e.g., at least 0.038 inch).

In some embodiments, outer diameter OD1 can be selected based on the size of the delivery system that will be used to deliver embolic coil 10 (e.g., a catheter having a certain inner diameter).

When embolic coil 10 is in its primary shape, embolic coil body 12 has a length L2 that is equal to length L1 of embolic coil 10, an inner diameter ID2 that is equal to inner diameter ID1 of embolic coil 10, and an outer diameter OD2. In some embodiments, outer diameter OD2 can be at least 0.0025 inch (e.g., at least 0.005 inch, at least 0.01 inch, at least 0.02 inch, at least 0.03 inch) and/or at most 0.0718 inch (e.g., at most 0.05 inch, at most 0.03 inch, at most 0.02 inch, at most 0.01 inch).

The pitch of an embolic coil body, such as embolic coil body 12, is the sum of the thickness of one winding of the embolic coil body (e.g., winding 15) and the amount of space between that winding and a consecutive winding of the embolic coil body (e.g., winding 16). FIG. 1C shows the pitch P1 of embolic coil body 12. In some embodiments, pitch P1 can be at most 0.015 inch (e.g., at most 0.01 inch, at most 0.005 inch, at most 0.003 inch, at most 0.002 inch) and/or at least 0.005 inch (e.g., at least 0.01 inch, at least 0.02 inch, at least 0.03 inch, at least 0.004 inch). Because the windings of embolic coil body 12 are flush with each other, pitch P1 is equal to the thickness of a winding of embolic coil body 12. However, in certain embodiments, an embolic coil body can include windings that are not flush with each other and that have space between them.

In general, an embolic coil such as embolic coil 10 has a primary shape and a secondary shape. Embolic coil 10 exhibits only its primary shape when embolic coil 10 is extended within lumen 105 of catheter 106 (as shown in FIG. 2C). As embolic coil 10 exits catheter 106, however, embolic coil 10 further assumes its secondary shape, which allows embolic coil 10 to fill aneurysmal sac 104. Typically, the primary shape of embolic coil 10 can be selected for deliverability, and the secondary shape of embolic coil 10 can be selected for application (e.g., embolization of an aneurysm).

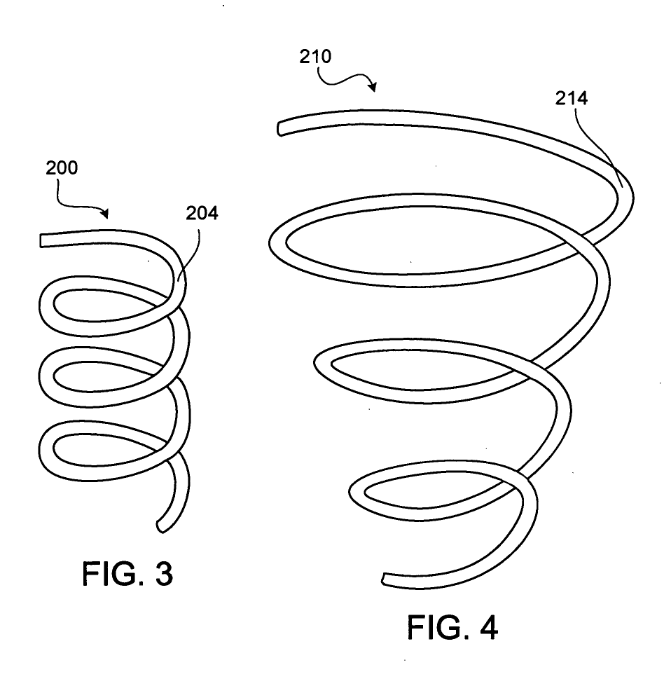

As FIGS. 3-9 illustrate, an embolic coil can have any of a number of different secondary shapes, which can depend on the particular application for the embolic coil.

As an example, FIG. 3 shows an embolic coil 200 having a spiral secondary shape. Embolic coil 200 includes a coating 204. An embolic coil with a spiral secondary shape can be used, for example, to provide a supportive framework along a vessel wall. Alternatively or additionally, an embolic coil with a spiral secondary shape can be used to hold other embolic coils that are subsequently delivered to the target site.

As another example, FIG. 4 shows an embolic coil 210 having a single apex vortex secondary shape (also known as a conical secondary shape). Embolic coil 210 includes a coating 214. An embolic coil with a single apex vortex secondary shape can be used, for example, to close the center of a target site (e.g., a vessel, an aneurysm) that is to be occluded, and/or to occlude a target site in conjunction with an embolic coil such as embolic coil 200 (FIG. 3). An embolic coil with a single apex vortex secondary shape can be used to occlude a vessel having low flow, intermediate low, or high flow. In some embodiments, multiple coils with single apex vortex secondary shapes can be used to occlude a vessel. In certain embodiments, an embolic coil with a single apex vortex secondary shape can be used as a packing coil, such that the coil can be packed into a vessel that is slightly smaller than the diameter of the coil. As an example, a six-millimeter diameter coil can be packed into a vessel having a five-millimeter diameter. In some embodiments, an embolic coil with a single apex vortex secondary shape can be used to embolize a tumor and and/or to treat gastrointestinal bleeding.

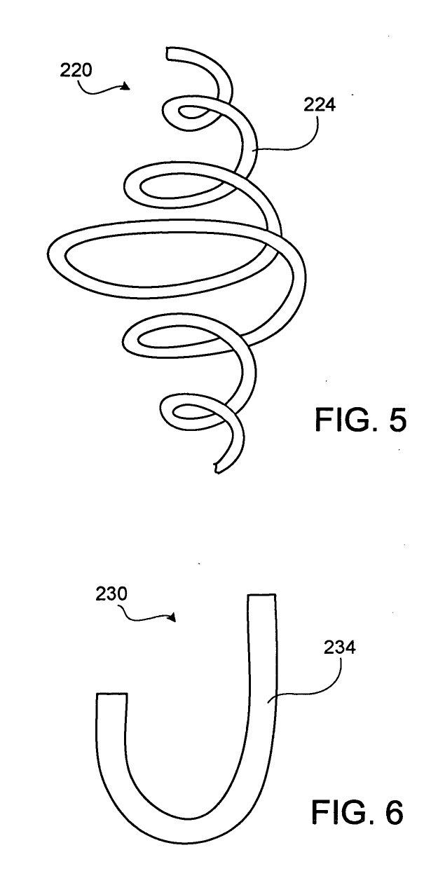

As an additional example, FIG. 5 shows an embolic coil 220 having a diamond secondary shape (also known as a double vortex secondary shape). Embolic coil 220 includes a coating 224. Like an embolic coil with a vortex secondary shape, an embolic coil with a diamond secondary shape can be used, for example, to close the center of a target site (e.g., a vessel, an aneurysm) that is to be occluded, and/or to occlude a target site in conjunction with an embolic coil such as embolic coil 200 (FIG. 3).

As a further example, FIG. 6 shows an embolic coil 230 having a secondary shape in the form of a J. Embolic coil 230 includes a coating 234. An embolic coil having a secondary shape in the form of a J can be used, for example, to fill remaining space in an aneurysm that was not filled by other coils. In some embodiments, an operator (e.g., a physician) can hook the curved portion of embolic coil 230 into a coil or coil mass that has already been deployed at a target site, and then shape the straighter portion of embolic coil 230 to fill the target site.

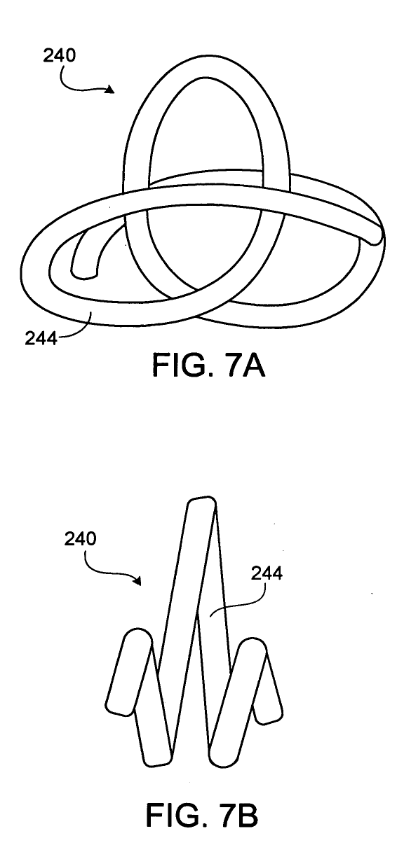

As another example, FIGS. 7A and 7B show an embolic coil 240 having a complex helical secondary shape. Embolic coil 240 includes a coating 244. An embolic coil with a complex helical secondary shape can be used, for example, to frame a target site. In certain embodiments, an embolic coil with a complex helical secondary shape can be used as an anchoring coil that helps to hold other embolic coils in place at a target site (e.g., thereby allowing additional embolic coils to be packed into the target site).

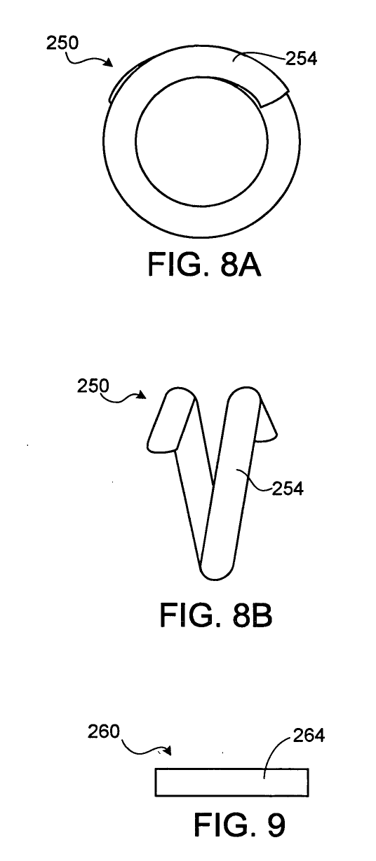

As an additional example, FIGS. 8A and 8B show an embolic coil 250 having a helical secondary shape. Embolic coil 250 includes a coating 254. An embolic coil with a helical secondary shape can be used, for example, as a packing coil.

As a further example, FIG. 9 shows an embolic coil 260 having a straight secondary shape. An embolic coil with a straight secondary shape can be used, for example, in a relatively small vessel (e.g., to block blood flow to a tumor).

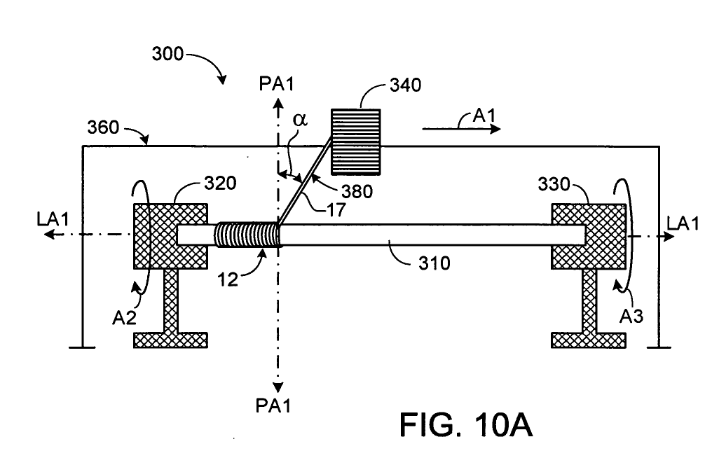

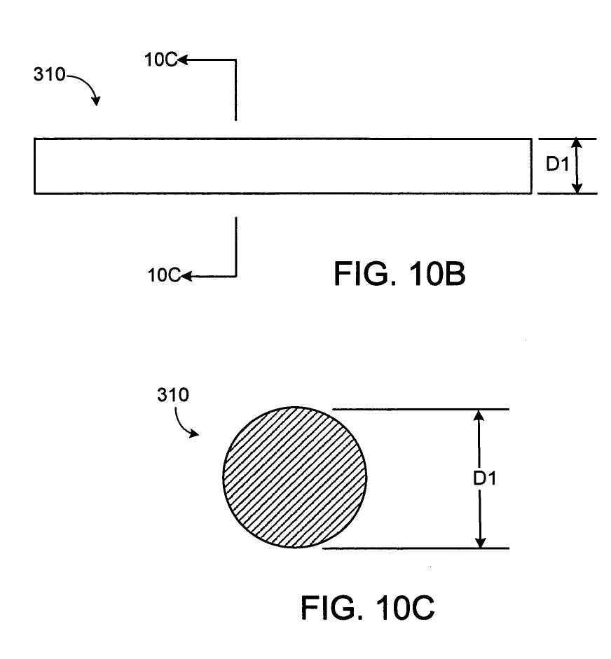

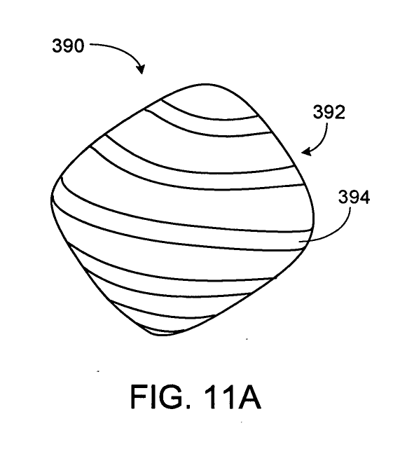

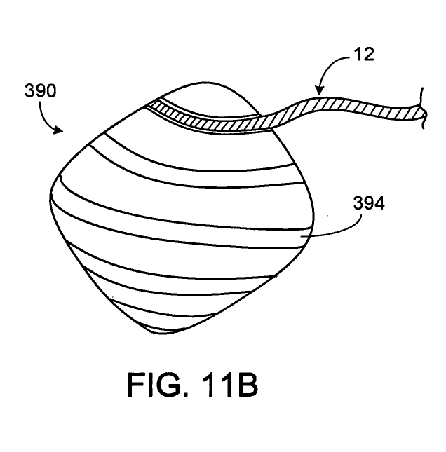

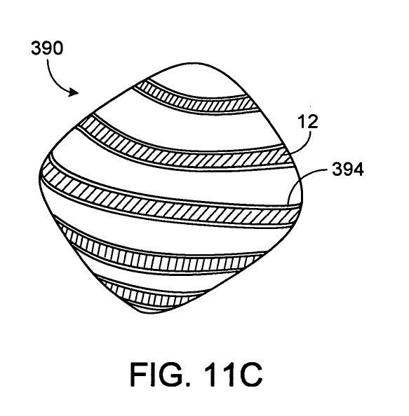

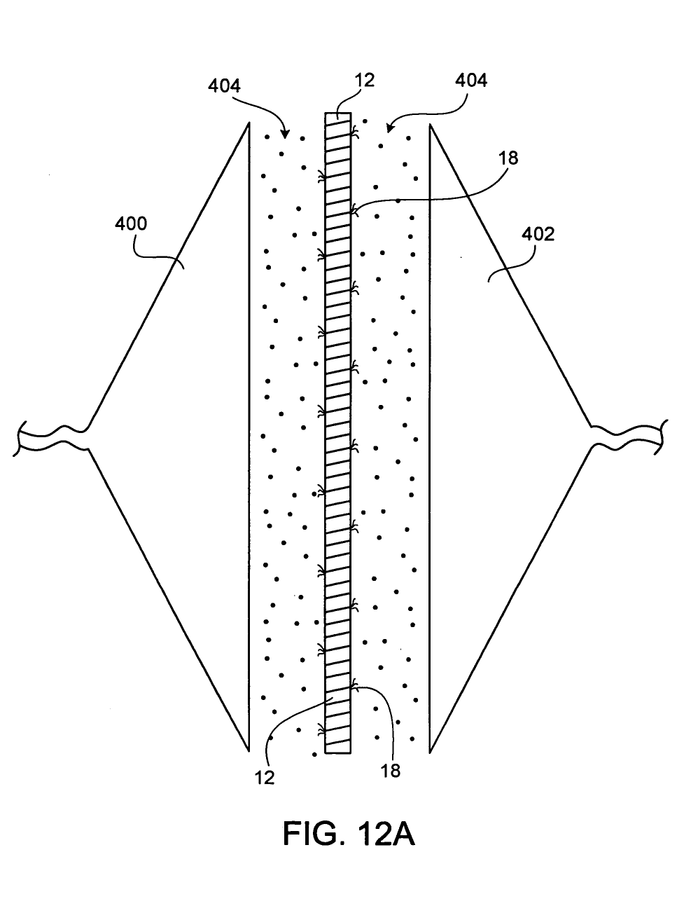

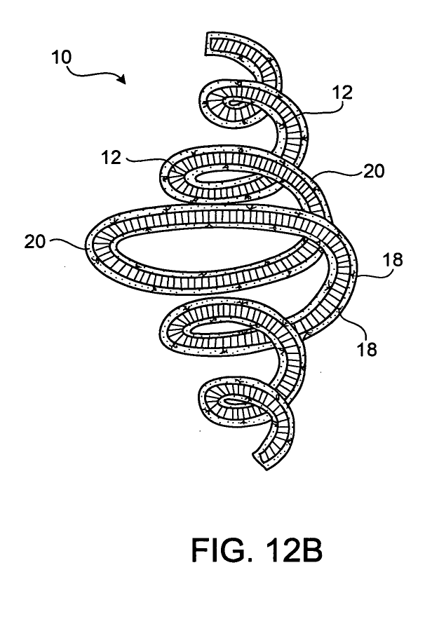

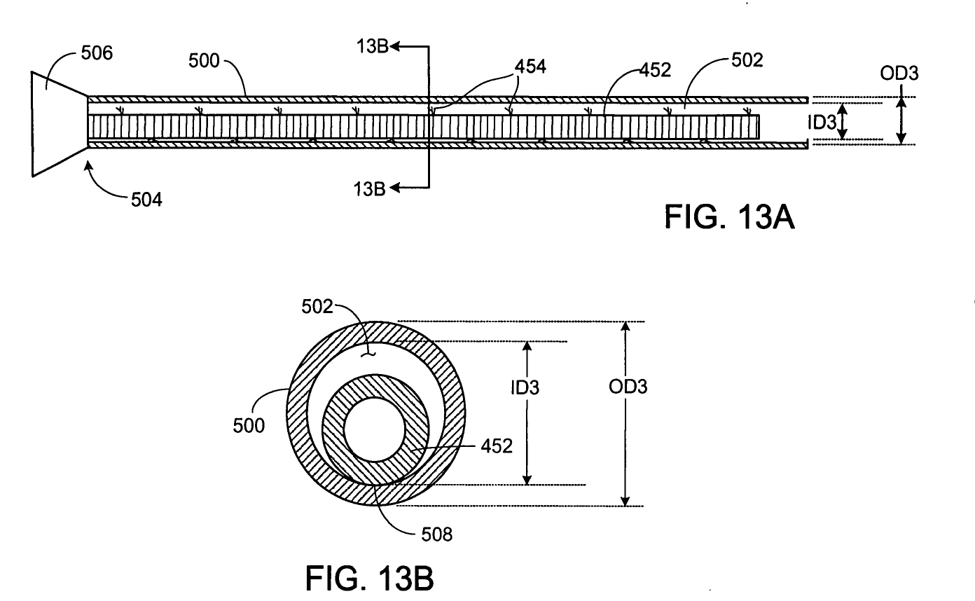

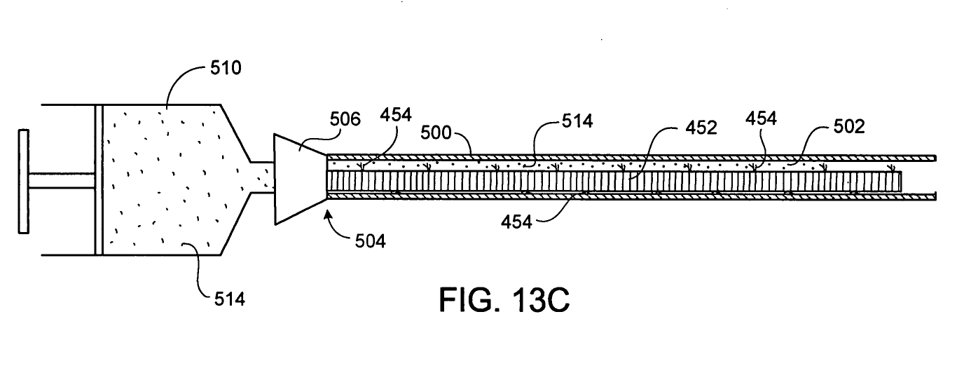

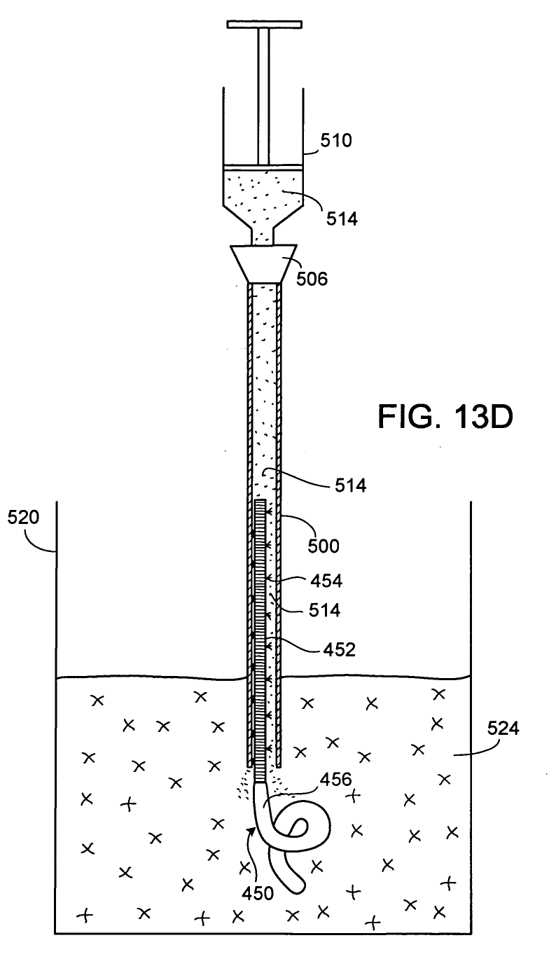

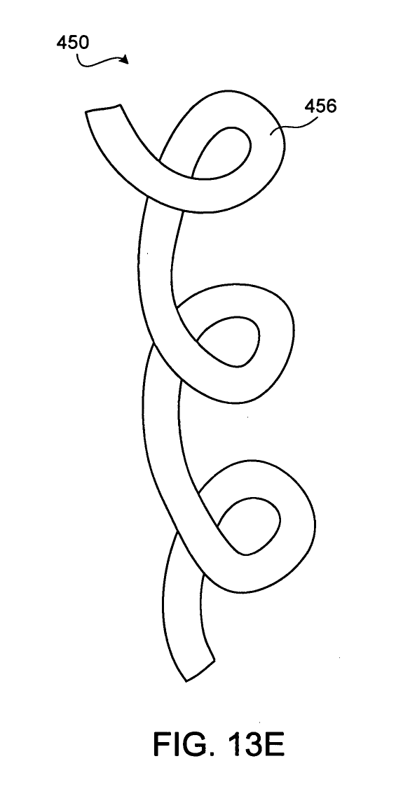

FIGS. 10A-10C illustrate a process and a mandrel used to form an embolic coil body in its primary shape, FIGS. 11A-11C illustrate a process and a mandrel used to shape the embolic coil body into a secondary shape, and FIGS. 12A and 13A-13D illustrate processes for coating an embolic coil body to form a coated embolic coil (e.g., embolic coil 10).

As shown in FIG. 10A, a coil-forming apparatus 300 includes a mandrel 310 held by two rotatable chucks 320 and 330. A spool 340 of wire 17 is disposed above mandrel 310, and is attached to a linear drive 360. To form an embolic coil in its primary shape, chucks 320 and 330 are activated so that they rotate in the direction of arrows A2 and A3, thereby rotating mandrel 310. Linear drive 360 also is activated, and moves spool 340 in the direction of arrow A1. The rotation of mandrel 310 pulls wire 17 from spool 340 at a predetermined pull-off angle (alpha) α, and causes wire 17 to wrap around mandrel 310, forming embolic coil body 12. The pull-off angle (alpha) α is the angle between axis PA1, which is perpendicular to longitudinal axis LA1 of mandrel 310, and the portion 380 of wire 17 between spool 340 and embolic coil body 12. In some embodiments, αcan be from about one degree to about six degrees (e.g., from about 1.5 degrees to about five degrees, from about 1.5 degrees to about 2.5 degrees, about two degrees). In certain embodiments, a controller (e.g., a programmable logic controller) can be used to maintain the pull-off angle (alpha) α in coil-forming apparatus 300. Because mandrel 310 is rotating as it is pulling wire 17 from spool 340, and because linear drive 360 is moving spool 340 in the direction of arrow A1, wire 17 forms embolic coil body 12 in a primary shape around mandrel 310. Embolic coil body 12 can be formed, for example, at room temperature (25° C.).

After embolic coil body 12 has been formed, chucks 320 and 330, and linear drive 360, are deactivated, and portion 380 of wire 17 is cut. Mandrel 310 is then released from chuck 320, and embolic coil body 12 is pulled off of mandrel 310. While embolic coil body 12 might lose some of its primary shape as it is pulled off of mandrel 310, embolic coil body 12 can generally return to its primary shape shortly thereafter, because of memory imparted to embolic coil body 12 during formation. In some embodiments, after embolic coil body 12 has been removed from mandrel 310, one or both of the ends of embolic coil body 12 can be heated and melted to form rounder, more biocompatible (e.g., atraumatic) ends.

Mandrel 310 can be formed of, for example, a metal or a metal alloy, such as stainless steel. In some embodiments, mandrel 310 can be formed of one or more polymers, such as Teflon® (polytetrafluoroethylene) or Delrin® (polyoxymethylene). In certain embodiments, mandrel 310 can be formed of a shape-memory material, such as Nitinol.

Mandrel 310 has a diameter D1 (FIGS. 10B and 10C). Diameter D1 can typically be selected based on the size of the coil to be formed using mandrel 310. In some embodiments, diameter D1 can be at least 0.0005 inch and/or at most 0.07 inch.

The tension of mandrel 310 as it is held between chucks 320 and 330 preferably is sufficiently high to avoid vibration of mandrel 310 during the winding process, and sufficiently low to avoid stretching of mandrel 310 during the winding process. In some instances, significant stretching of mandrel 310 during the winding process could cause embolic coil body 12 to have a smaller primary shape than desired, and/or could make it relatively difficult to remove embolic coil body 12 from mandrel 310. In certain embodiments, the tension of mandrel 310 can be from about 100 grams to about 1,000 grams (e.g., from about 300 grams to about 600 grams, from about 400 grams to about 500 grams). For example, the tension of mandrel 310 can be about 506 grams.

In some embodiments, wire 17 can be wound around mandrel 310 at a tension of at least about four grams (e.g., at least about five grams, at least about six grams, at least about 10 grams, at least about 22 grams, at least about 27 grams, at least about 32 grams, at least about 40 grams, at least about 60 grams, at least about 65 grams, at least about 85 grams) and/or at most about 100 grams (e.g., at most about 85 grams, at most about 65 grams, at most about 60 grams, at most about 40 grams, at most about 32 grams, at most about 27 grams, at most about 22 grams, at most about 10 grams, at most about six grams, at most about five grams).

In certain embodiments, the length of embolic coil body 12 in its primary shape and while under tension on mandrel 310 can be from about 10 centimeters to about 250 centimeters (e.g., from about 50 centimeters to about 200 centimeters, from about 130 centimeters to about 170 centimeters, from about 144 centimeters to about 153 centimeters, from about 147 centimeters to about 153 centimeters). For example, the length of embolic coil body 12 in its primary shape and while under tension on mandrel 310 can be about 132 centimeters or about 147 centimeters. Embolic coil body 12 may recoil to some extent (e.g., by at most about five centimeters) when portion 380 of wire 17 is severed, such that embolic coil body 12 will be somewhat smaller once it has been removed from mandrel 310. In some embodiments, embolic coil body 12 can have a length of from about five centimeters to about 225 centimeters (e.g., from about 25 centimeters to about 170 centimeters, from about 120 centimeters to about 140 centimeters, from about 137 centimeters to about 140 centimeters) after being removed from mandrel 310. After embolic coil body 12 has been removed from mandrel 310, embolic coil body 12 can be cut into smaller coils.

Once embolic coil body 12 has been formed in its primary shape, embolic coil body 12 can be further shaped into a secondary shape, as shown in FIGS. 11A-11C.

FIG. 11A shows a mandrel 390 used to form the secondary shape of embolic coil body 12. While mandrel 390 is shaped to form a diamond (also known as a double vortex), other types of mandrels can be used to form other secondary shapes. Mandrel 390 is formed of a diamond-shaped block 392 with grooves 394 cut into its surface. As shown in FIGS. 11B and 11C, embolic coil body 12 in its primary shape is wrapped around mandrel 390, such that embolic coil body 12 fills grooves 394, creating the secondary shape. The ends of embolic coil body 12 are then attached (e.g., pinned) to mandrel 390, and embolic coil body 12 is heat-treated to impart memory to coil body 12. In some embodiments, embolic coil body 12 can be heat-treated at a temperature of at least about 1000° C. (e.g., at least about 1050° C., at least about 1100° C., at least about 1150° C.), and/or at most about 1200° C. (e.g., at most about 1150° C., at most about 1100° C., at most about 1050° C.). In certain embodiments, the heat treatment of embolic coil body 12 can last for a period of from about 10 minutes to about 40 minutes (e.g., about 25 minutes). After being heat-treated, embolic coil body 12 is unwrapped from mandrel 390. The removal of embolic coil body 12 from mandrel 390 allows embolic coil body 12 to reassume its secondary shape. In some embodiments, after embolic coil body 12 has been removed from mandrel 390, one or both of the ends of embolic coil body 12 can be heated and melted to form rounder, more atraumatic ends.

Mandrel 390 can be formed of, for example, a metal or a metal alloy (e.g., stainless steel). In some embodiments, mandrel 390 can be formed of a plated metal or a plated metal alloy (e.g., chrome-plated stainless steel).

After embolic coil body 12 has been removed from mandrel 390, fibers can be attached to embolic coil body 12. In certain embodiments, embolic coil body 12 can be stretched prior to attaching fibers, so that embolic coil body 12 is in its primary shape, and can then be loaded onto a fibering mandrel (e.g., a fibering mandrel from Sematool Mold and Die Co., Santa Clara, Calif.). In some embodiments, fibers can be attached to embolic coil body 12 by tying the fibers to wire 17 of embolic coil body 12, wrapping the fibers around wire 17, and/or snapping the fibers in between windings of wire 17. In certain embodiments, one portion (e.g., one end) of a bunch of fibers can be snapped in between windings in one region of embolic coil body 12, and another portion (e.g., the other end) of the same bunch of fibers can be wrapped around part of embolic coil body 12 and snapped in between windings in another region of embolic coil body 12. In some embodiments, fibers can be attached to embolic coil body 12 by bonding (e.g., adhesive bonding) the fibers to wire 17 of embolic coil body 12.

FIG. 12A illustrates an embodiment of a process that can be used to coat embolic coil body 12 after embolic coil body 12 has been fibered. As shown in FIG. 12A, embolic coil body 12 is restrained in its primary shape and placed between two sprayers 400 and 402 that spray a coating material 404 onto embolic coil body 12. In some embodiments, the viscosity of coating material 404 can be selected so that coating material 404 remains on embolic coil body 12. In certain embodiments, coating material 404 can have a viscosity of at least about one centipoise and/or at most about 400 centipoise, as measured using a digital cone/plate viscometer from Brookfield Engineering at a temperature of from 65° C. to 75° C. and a spindle speed of from 1.5 rpm to 3.0 rpm. After embolic coil body 12 has been sprayed with coating material 404 and allowed to re-assume its secondary shape, the result, as shown in FIG. 12B, is embolic coil 10, including coating 20.

While FIG. 12A illustrates a method of coating embolic coil body 12 after embolic coil body 12 has been formed into its secondary shape and fibered, in some embodiments, other methods can be used to form a coated coil. As an example, in certain embodiments, embolic coil body 12 can be coated prior to being formed into a secondary shape. As another example, in some embodiments, wire 17 can include a coating. Thus, when wire 17 is used to form embolic coil body 12, embolic coil body 12 can also include the coating. Wire 17 can be coated using, for example, one or more spray coating methods and/or dip coating methods.

While FIG. 12A shows one method of coating an embolic coil body to form a coated embolic coil, other methods can be used. For example, FIGS. 13A-13D illustrate a method of forming a coated embolic coil 450 (FIG. 13E).

As shown in FIGS. 13A and 13B, an embolic coil body 452 in its primary shape is placed into a lumen 502 of an introducer sheath 500. Fiber bundles 454 are attached to embolic coil body 452. Introducer sheath 500 has an inner diameter ID3 and an outer diameter OD3. In some embodiments, inner diameter ID3 can be at least 0.0027 inch (e.g., at least 0.01 inch) and/or at most 0.1 inch (e.g., at most 0.04 inch). In certain embodiments, outer diameter OD3 can be at least 0.02 inch (e.g., at least 0.035 inch) and/or at most 0.1 inch (e.g., at most 0.072 inch).

At its proximal end 504, introducer sheath 500 is connected to a female luer lock component 506. As shown in FIG. 13B, embolic coil body 452 is not suspended within lumen 502. Rather, embolic coil body 452 is in some contact with a wall 508 of introducer sheath 500.

As FIG. 13C shows, a syringe 510 containing a solution 514 including a gelling precursor (e.g., alginate) is connected to introducer sheath 500 via female luer lock component 506. Solution 514 is partially injected into lumen 502 of introducer sheath 500, so that solution 514 contacts embolic coil body 452 and fiber bundles 454. As shown in FIG. 13D, after solution 514 has flowed over at least a portion of embolic coil body 452 and fiber bundles 454, syringe 510 is used to inject both solution 514 and embolic coil body 452 into a vessel 520 containing a solution 524 including a gelling agent (e.g., calcium chloride). As embolic coil body 452 is delivered into solution 524, the interaction between solution 514 and solution 524 at the surface of embolic coil body 452 and on fiber bundles 454 results in the formation of a coated coil 450 (FIG. 13E). Coated coil 450 includes a gel coating 456 formed of the gelled gelling precursor.

While certain methods of coating an embolic coil body to form a coated embolic coil have been described, in some embodiments, other methods can be used. As an example, in certain embodiments, a dip-coating process can be used to coat an embolic coil body.

Embolic coils and methods of making embolic coils are described, for example, in Elliott et al., U.S. Patent Application Publication No. US 2006/0116711 A1, published on Jun. 1, 2006, and entitled “Embolic Coils”; Buiser et al., U.S. patent application Ser. No. 11/311,617, filed on Dec. 19, 2005, and entitled “Coils”; and Buiser et al., U.S. patent application Ser. No. 11/430,602, filed on May 9, 2006, and entitled “Embolic Coils”, all of which are incorporated herein by reference. Methods of forming gels are described, for example, in Lanphere et al., U.S. Patent Application Publication No. US 2004/0096662 A1, published on May 20, 2004, and entitled “Embolization”, and in DiCarlo et al., U.S. patent application Ser. No. 11/111,511, filed on Apr. 21, 2005, and entitled “Particles”, both of which are incorporated herein by reference.

In some embodiments, an embolic coil such as embolic coil 10 can include one or more therapeutic agents (e.g., drugs). For example, embolic coil body 12, fiber bundles 18, and/or coating 20 of embolic coil 10 can include one or more therapeutic agents. Embolic coil 10 can, for example, be used to deliver the therapeutic agents to a target site.

In certain embodiments, one component of embolic coil 10 (e.g., embolic coil body 12) can include one or more therapeutic agents that are the same as, or different from, one or more therapeutic agents in coating 20. In some embodiments, therapeutic agents can be dispersed within coating 12. In certain embodiments, coating 12 can contain a therapeutic agent (e.g., heparin) that limits or prevents thrombosis. When coating 12 is eroded and/or absorbed, thereby releasing the therapeutic agent into the body of the subject (e.g., during delivery), the therapeutic agent can limit or prevent premature thrombosis.

In some embodiments, embolic coil 10 can include one or more therapeutic agents that are coated onto embolic coil body 12, and/or that are coated onto coating 20. In some embodiments, a therapeutic agent can be compounded with a polymer that is included in coating 20. In certain embodiments, a therapeutic agent can be applied to the surface of embolic coil body 12 and/or to coating 20 by exposing embolic coil body 12 and/or coating 20 to a high concentration solution of the therapeutic agent.

In some embodiments, a therapeutic agent-coated embolic coil can include a coating (e.g., a bioerodible and/or bioabsorbable polymer coating) over the surface the therapeutic agent. The coating can assist in controlling the rate at which therapeutic agent is released from the embolic coil. For example, the coating can be in the form of a porous membrane. The coating can delay an initial burst of therapeutic agent release. The coating can be applied by dipping or spraying the embolic coil. The coating can include therapeutic agent or can be substantially free of therapeutic agent. The therapeutic agent in the coating can be the same as or different from an agent on a surface layer of the embolic coil body, and/or in a coating on the embolic coil body, and/or within the embolic coil body. A polymer coating (e.g., that is bioerodible and/or bioabsorbable) can be applied to an embolic coil body surface and/or to a coated embolic coil surface in embodiments in which a high concentration of therapeutic agent has not been applied to the embolic coil body surface or to the coated coil surface.

Coatings are described, for example, in DiMatteo et al., U.S. Patent Application Publication No. US 2004/0076582 A1, published on Apr. 22, 2004, and entitled “Agent Delivery Particle”, which is incorporated herein by reference.

In some embodiments, one or more embolic coils can be disposed in a therapeutic agent that can serve as a pharmaceutically acceptable carrier.

Therapeutic agents include genetic therapeutic agents, non-genetic therapeutic agents, and cells, and can be negatively charged, positively charged, amphoteric, or neutral. Therapeutic agents can be, for example, materials that are biologically active to treat physiological conditions; pharmaceutically active compounds; gene therapies; nucleic acids with and without carrier vectors (e.g., recombinant nucleic acids, DNA (e.g., naked DNA), cDNA, RNA, genomic DNA, cDNA or RNA in a non-infectious vector or in a viral vector which may have attached peptide targeting sequences, antisense nucleic acids (RNA, DNA)); peptides (e.g., growth factor peptides, such as basic fibroblast growth factor (bFGF)); oligonucleotides; gene/vector systems (e.g., anything that allows for the uptake and expression of nucleic acids); DNA chimeras (e.g., DNA chimeras which include gene sequences and encoding for ferry proteins such as membrane translocating sequences (“MTS”) and herpes simplex virus-1 (“VP22”)); compacting agents (e.g., DNA compacting agents); viruses; polymers; hyaluronic acid; proteins (e.g., enzymes such as ribozymes, asparaginase); immunologic species; nonsteroidal anti-inflammatory medications; chemoagents; pain management therapeutics; oral contraceptives; progestins; gonadotrophin-releasing hormone agonists; chemotherapeutic agents; and radioactive species (e.g., radioisotopes, radioactive molecules). Non-limiting examples of therapeutic agents include anti-thrombogenic agents; antioxidants; angiogenic and anti-angiogenic agents and factors; anti-proliferative agents (e.g., agents capable of blocking smooth muscle cell proliferation); calcium entry blockers; and survival genes which protect against cell death (e.g., anti-apoptotic Bcl-2 family factors and Akt kinase).

Exemplary non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, acetyl salicylic acid, sulfasalazine and mesalamine; antineoplastic/antiproliferative/anti-mitotic agents such as paclitaxel, 5-fluorouracil, cisplatin, methotrexate, doxorubicin, vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet factors or peptides; vascular cell growth promoters such as growth factors, transcriptional activators, and translational promoters; vascular cell growth inhibitors such as growth factor inhibitors (e.g., PDGF inhibitor-Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Exemplary genetic therapeutic agents include: anti-sense DNA and RNA; DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or additionally, molecules capable of inducing an upstream or downstream effect of a BMP can be provided. Such molecules include any of the “hedgehog” proteins, or the DNA's encoding them. Vectors of interest for delivery of genetic therapeutic agents include: plasmids; viral vectors such as adenovirus (AV), adenoassociated virus (AAV) and lentivirus; and non-viral vectors such as lipids, liposomes and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents appropriate for the practice of the present invention are disclosed in Kunz et al., U.S. Pat. No. 5,733,925, assigned to NeoRx Corporation, which is incorporated herein by reference. Therapeutic agents disclosed in this patent include the following:

“Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

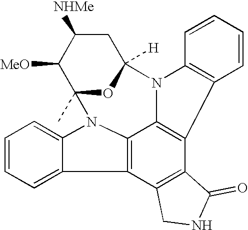

as well as diindoloalkaloids having one of the following general structures: 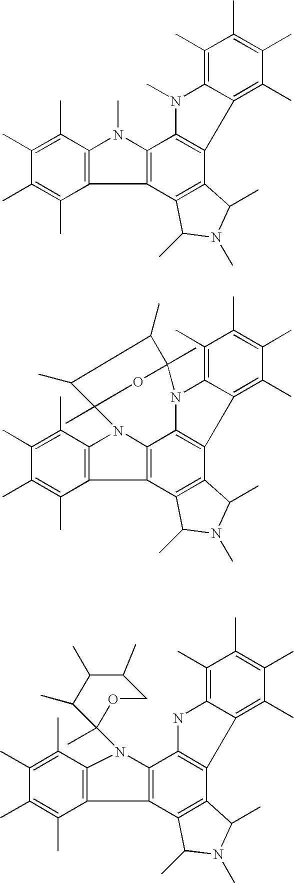
as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like. Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell), such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents include one or more of the following: calcium-channel blockers, including benzothiazapines (e.g., diltiazem, clentiazem); dihydropyridines (e.g., nifedipine, amlodipine, nicardapine); phenylalkylamines (e.g., verapamil); serotonin pathway modulators, including 5-HT antagonists (e.g., ketanserin, naftidrofuryl) and 5-HT uptake inhibitors (e.g., fluoxetine); cyclic nucleotide pathway agents, including phosphodiesterase inhibitors (e.g., cilostazole, dipyridamole), adenylate/guanylate cyclase stimulants (e.g., forskolin), and adenosine analogs; catecholamine modulators, including α-antagonists (e.g., prazosin, bunazosine), β-antagonists (e.g., propranolol), and α/β-antagonists (e.g., labetalol, carvedilol); endothelin receptor antagonists; nitric oxide donors/releasing molecules, including organic nitrates/nitrites (e.g., nitroglycerin, isosorbide dinitrate, amyl nitrite), inorganic nitroso compounds (e.g., sodium nitroprusside), sydnonimines (e.g., molsidomine, linsidomine), nonoates (e.g., diazenium diolates, NO adducts of alkanediamines), S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), C-nitroso-, O-nitroso- and N-nitroso-compounds, and L-arginine; ACE inhibitors (e.g., cilazapril, fosinopril, enalapril); ATII-receptor antagonists (e.g., saralasin, losartin); platelet adhesion inhibitors (e.g., albumin, polyethylene oxide); platelet aggregation inhibitors, including aspirin and thienopyridine (ticlopidine, clopidogrel) and GP IIb/IIIa inhibitors (e.g., abciximab, epitifibatide, tirofiban, intergrilin); coagulation pathway modulators, including heparinoids (e.g., heparin, low molecular weight heparin, dextran sulfate, β-cyclodextrin tetradecasulfate), thrombin inhibitors (e.g., hirudin, hirulog, PPACK (D-phe-L-propyl-L-arg-chloromethylketone), argatroban), FXa inhibitors (e.g., antistatin, TAP (tick anticoagulant peptide)), vitamin K inhibitors (e.g., warfarin), and activated protein C; cyclooxygenase pathway inhibitors (e.g., aspirin, ibuprofen, flurbiprofen, indomethacin, sulfinpyrazone); natural and synthetic corticosteroids (e.g., dexamethasone, prednisolone, methprednisolone, hydrocortisone); lipoxygenase pathway inhibitors (e.g., nordihydroguairetic acid, caffeic acid; leukotriene receptor antagonists; antagonists of E- and P-selectins; inhibitors of VCAM-1 and ICAM-1 interactions; prostaglandins and analogs thereof, including prostaglandins such as PGE1 and PGI2; prostacyclin analogs (e.g., ciprostene, epoprostenol, carbacyclin, iloprost, beraprost); macrophage activation preventers (e.g., bisphosphonates); HMG-CoA reductase inhibitors (e.g., lovastatin, pravastatin, fluvastatin, simvastatin, cerivastatin); fish oils and omega-3-fatty acids; free-radical scavengers/antioxidants (e.g., probucol, vitamins C and E, ebselen, retinoic acid (e.g., trans-retinoic acid), SOD mimics); agents affecting various growth factors including FGF pathway agents (e.g., bFGF antibodies, chimeric fusion proteins), PDGF receptor antagonists (e.g., trapidil), IGF pathway agents (e.g., somatostatin analogs such as angiopeptin and ocreotide), TGF-β pathway agents such as polyanionic agents (heparin, fucoidin), decorin, and TGF-β antibodies, EGF pathway agents (e.g., EGF antibodies, receptor antagonists, chimeric fusion proteins), TNF-α pathway agents (e.g., thalidomide and analogs thereof), thromboxane A2 (TXA2) pathway modulators (e.g., sulotroban, vapiprost, dazoxiben, ridogrel), protein tyrosine kinase inhibitors (e.g., tyrphostin, genistein, and quinoxaline derivatives); MMP pathway inhibitors (e.g., marimastat, ilomastat, metastat), and cell motility inhibitors (e.g., cytochalasin B); antiproliferative/antineoplastic agents including antimetabolites such as purine analogs (e.g., 6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin, daunomycin, bleomycin, mitomycin, penicillins, cephalosporins, ciprofalxin, vancomycins, aminoglycosides, quinolones, polymyxins, erythromycins, tertacyclines, chloramphenicols, clindamycins, linomycins, sulfonamides, and their homologs, analogs, fragments, derivatives, and pharmaceutical salts), nitrosoureas (e.g., carmustine, lomustine) and cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin and squalamine), and rapamycin, cerivastatin, flavopiridol and suramin; matrix deposition/organization pathway inhibitors (e.g., halofuginone or other quinazolinone derivatives, tranilast); endothelialization facilitators (e.g., VEGF and RGD peptide); and blood rheology modulators (e.g., pentoxifylline).

Other examples of therapeutic agents include anti-tumor agents, such as docetaxel, alkylating agents (e.g., mechlorethamine, chlorambucil, cyclophosphamide, melphalan, ifosfamide), plant alkaloids (e.g., etoposide), inorganic ions (e.g., cisplatin), biological response modifiers (e.g., interferon), and hormones (e.g., tamoxifen, flutamide), as well as their homologs, analogs, fragments, derivatives, and pharmaceutical salts.

Additional examples of therapeutic agents include organic-soluble therapeutic agents, such as mithramycin, cyclosporine, and plicamycin. Further examples of therapeutic agents include pharmaceutically active compounds, anti-sense genes, viral, liposomes and cationic polymers (e.g., selected based on the application), biologically active solutes (e.g., heparin), prostaglandins, prostcyclins, L-arginine, nitric oxide (NO) donors (e.g., lisidomine, molsidomine, NO-protein adducts, NO-polysaccharide adducts, polymeric or oligomeric NO adducts or chemical complexes), enoxaparin, Warafin sodium, dicumarol, interferons, chymase inhibitors (e.g., Tranilast), ACE inhibitors (e.g., Enalapril), serotonin antagonists, 5-HT uptake inhibitors, and beta blockers, and other antitumor and/or chemotherapy drugs, such as BiCNU, busulfan, carboplatinum, cisplatinum, cytoxan, DTIC, fludarabine, mitoxantrone, velban, VP-16, herceptin, leustatin, navelbine, rituxan, and taxotere.

Therapeutic agents are described, for example, in DiMatteo et al., U.S. Patent Application Publication No. US 2004/0076582 A1, published on Apr. 22, 2004, and entitled “Agent Delivery Particle”, in Pinchuk et al., U.S. Pat. No. 6,545,097, and in Schwarz et al., U.S. Pat. No. 6,368,658, all of which are incorporated herein by reference.

While certain embodiments have been described, other embodiments are possible.

As an example, in some embodiments, an embolic coil with a coil body and one or more fiber bundles can be coated in certain sections and not in other sections.

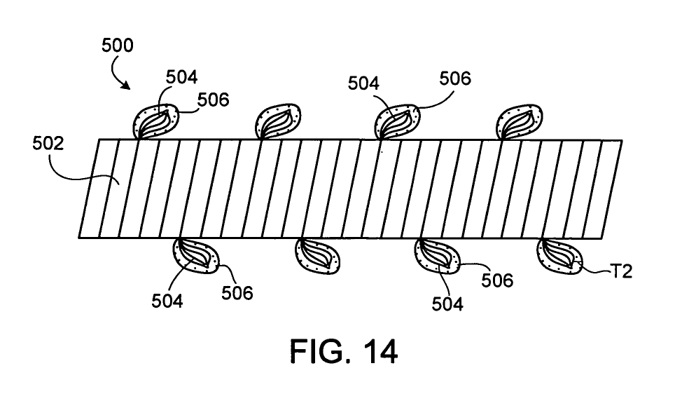

For example, FIG. 14 shows an embolic coil 500 including an embolic coil body 502, and fiber bundles 504 that are coated with a coating material 506 having a thickness T2. In some embodiments, thickness T2 can be at least 0.0001 inch (e.g., at least 0.001 inch, at least 0.002 inch) and/or at most 0.0345 inch (e.g., at most 0.02 inch, at most 0.002 inch, at most 0.001 inch). As FIG. 14 shows, while fiber bundles 504 are coated with coating material 506, embolic coil body 502 is not coated with coating material 506, or with any other coating material. Fiber bundles 504 can be coated with coating material 506 prior to, during, and/or after attachment of fiber bundles 504 to embolic coil body 502. In some embodiments, fiber bundles 504 can be formed from a spool of fiber material that has been coated with coating material 506.

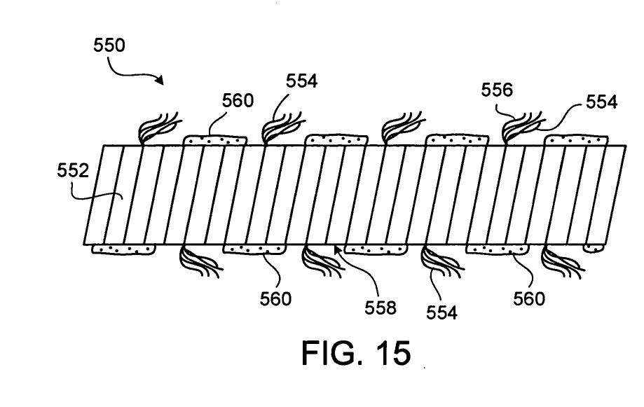

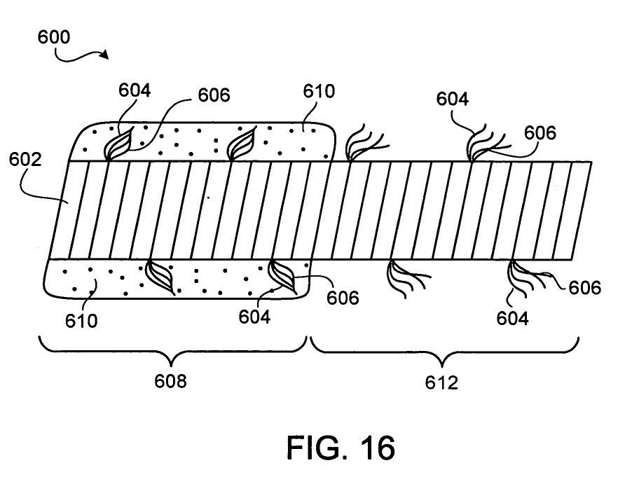

FIG. 15 shows an embolic coil 550 including an embolic coil body 552 and fiber bundles 554 formed of fibers 556. As FIG. 15 shows, certain regions of the exterior surface 558 of embolic coil body 552 are coated with a coating material 560. However, fiber bundles 554 are not coated with coating material 560, or with any other coating material.

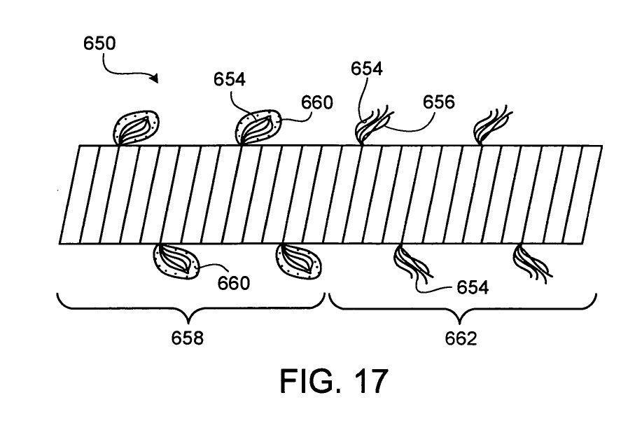

FIG. 16 shows an embolic coil 600 including an embolic coil body 602 and fiber bundles 604 formed of fibers 606. As FIG. 16 shows, one region 608 of embolic coil 600 includes a coating 610 over embolic coil body 602 and fiber bundles 604, while another region 612 of embolic coil 600 does not include a coating over embolic coil body 602 and fiber bundles 604.

FIG. 17 shows an embolic coil 650 including an embolic coil body 652 and fiber bundles 654 formed of fibers 656. As FIG. 17 shows, one region 658 of embolic coil 650 includes a coating material 660 over fiber bundles 654 (and not over embolic coil body 652), while another region 662 of embolic coil 650 does not include any coating material.

As an additional example, in some embodiments, an embolic coil can include one or more materials that can be dissolved by contact with an agent. For example, in certain embodiments, an embolic coil can include calcium alginate (e.g., in the form of a coating on the embolic coil body), which can be dissolved, for example, by contacting the embolic coil with sodium hexa-metaphosphate.

As another example, in some embodiments, an embolic coil can include multiple (e.g., two, three, four, five, 10, 20) different coatings. For example, in certain embodiments, an embolic coil can include an embolic coil body that is coated with one type of material, and fiber bundles that are coated with another, different, type of material.

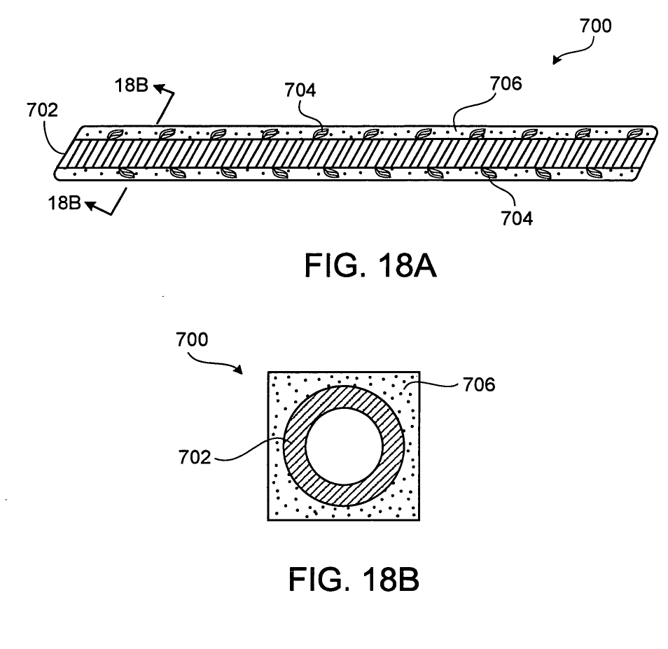

As an additional example, a coated embolic coil may have a circular cross-section or a non-circular cross-section, or may have a circular cross-section in one region and a non-circular cross-section in another region. For example, a coated embolic coil may have a polygonal cross-section (a non-circular cross-section that is a closed plane figure bounded by straight lines). As an example, FIGS. 18A and 18B show a coated embolic coil 700 including an embolic coil body 702, fiber bundles 704, and a coating 706 covering embolic coil body 702 and fiber bundles 704. Coated embolic coil 700 has a square cross-section. Coated embolic coils with non-circular (e.g., square) cross-sections are described, for example, in Buiser et al., U.S. patent application Ser. No. 11/311,617, filed on Dec. 19, 2005, and entitled “Coils”, which is incorporated herein by reference.

As a further example, in some embodiments, an embolic coil can include a porous coating. Embolic coils with porous coatings are described, for example, in Buiser et al., U.S. patent application Ser. No. 11/311,617, filed on Dec. 19, 2005, and entitled “Coils”, which is incorporated herein by reference. In certain embodiments, an embolic coil can include a non-porous coating. In some embodiments, an embolic coil can include both a porous coating and a non-porous coating.

As another example, while embolic coils including embolic coil bodies formed of windings of wire have been described, in some embodiments, an embolic coil can be formed of windings of a different substrate, such as a ribbon. Coils formed out of windings of ribbon are described, for example, in Buiser et al., U.S. patent application Ser. No. 11/430,602, filed on May 9, 2006, and entitled “Embolic Coils”, which is incorporated herein by reference.

As a further example, in some embodiments, an embolic coil including an embolic coil body and a coating can be stored in saline and/or deionized water, which can hydrate the coating.

As another example, in certain embodiments, a coated coil can be dried. Examples of methods that can be used to dry a coated coil include lyophilization, freeze-drying, and allowing the coil to dry in the air. In certain embodiments, a coated coil can be dried using a convection oven. A coated coil may be dried, for example, to enhance the attachment of a delivery wire to the coil, and/or to enhance loading of the coil into a sheath and/or other delivery device (e.g., a catheter). The dried coated coil can re-hydrate, for example, upon contacting a pharmaceutically acceptable carrier, and/or upon contacting body fluid after being delivered into a body of a subject.

As a further example, in some embodiments, an embolic coil can have at least two (e.g., three, four, five, 10, 15, 20) different outer diameters. Embolic coils with different outer diameters are described, for example, in Elliott et al., U.S. Patent Application Publication No. US 2006/0116711 A1, published on Jun. 1, 2006, and entitled “Embolic Coils”, and in Buiser et al., U.S. patent application Ser. No. 11/430,602, filed on May 9, 2006, and entitled “Embolic Coils”, both of which are incorporated herein by reference.

As an additional example, while embodiments have been shown in which the pitch of an embolic coil body is substantially the same in different regions of the embolic coil body, in certain embodiments, the pitch of an embolic coil body can differ in different regions of the embolic coil body. For example, some regions of an embolic coil body can have a pitch of 0.002 inch, while other regions of an embolic coil body can have a pitch of 0.004 inch.

As a further example, in some embodiments, an embolic coil can be a pushable embolic coil. The embolic coil can be delivered, for example, by pushing the embolic coil out of a delivery device (e.g., a catheter) using a pusher wire. Pushable embolic coils are described, for example, in Elliott et al., U.S. Patent Application Publication No. US 2006/0116711 A1, published on Jun. 1, 2006, and entitled “Embolic Coils”, and in Buiser et al., U.S. patent application Ser. No. 11/430,602, filed on May 9, 2006, and entitled “Embolic Coils”, both of which are incorporated herein by reference.

As another example, while an electrolytically detachable embolic coil has been shown, in some embodiments, an embolic coil can alternatively or additionally be a chemically detachable embolic coil and/or a mechanically detachable embolic coil. In certain embodiments, an embolic coil can be a Guglielmi Detachable Coil (GDC) or an Interlocking Detachable Coil (IDC) (e.g., a Fibered Interlocking Detachable Coil (FIDC)). Detachable embolic coils are described, for example, in Twyford, Jr. et al., U.S. Pat. No. 5,304,195; Guglielmi et al., U.S. Pat. No. 5,895,385; and Buiser et al., U.S. patent application Ser. No. 11/311,617, filed on Dec. 19, 2005, and entitled “Coils”, all of which are hereby incorporated by reference.

As a further example, in some embodiments, an embolic coil can be injectable. In certain embodiments, an injectable embolic coil can be disposed within a delivery device (e.g., a catheter) that is used to deliver the embolic coil to a target site. Once at the target site, the injectable embolic coil can be delivered into the target site using a high-pressure saline flush that pushes the embolic coil out the of the delivery device. In some embodiments, a pusher wire can be used in conjunction with a saline flush to deliver an embolic coil to a target site. In certain embodiments, a pusher wire may not be used in conjunction with a saline flush to deliver an embolic coil to a target site.

As an additional example, in certain embodiments, an embolic coil may be at least partially delivered from a delivery device, and then may be withdrawn back into the delivery device. In some embodiments in which the embolic coil includes a coating, the coating can enhance the withdrawal of the embolic coil back into the delivery device.

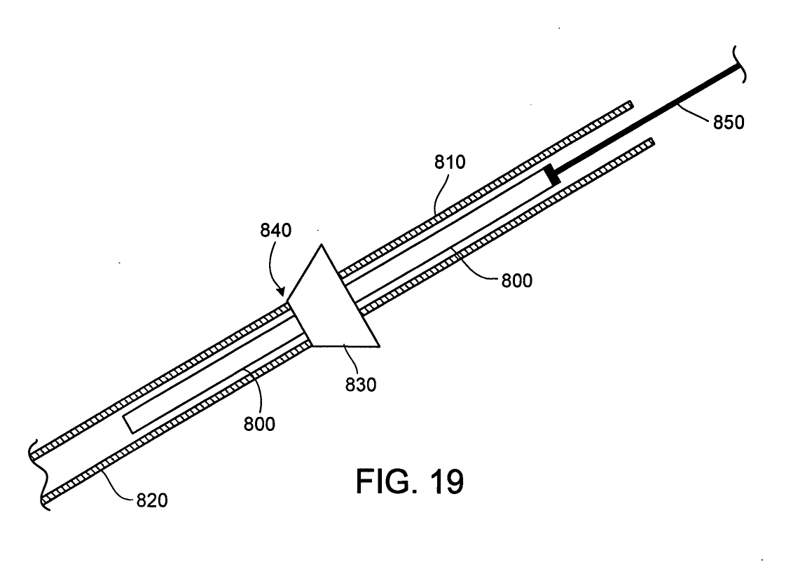

As another example, in certain embodiments, an embolic coil can be loaded into a delivery device using an introducer sheath. For example, FIG. 19 illustrates the transfer of an embolic coil 800 from an introducer sheath 810 into a catheter 820. A hub 830 located at the proximal end 840 of catheter 820 directs the placement of introducer sheath 810. After introducer sheath 810 has been placed in hub 830, a pusher 850 is used to push embolic coil 800 out of introducer sheath 810 and into catheter 820.

As an additional example, in some embodiments, a saline flush (e.g., a heparinized saline flush) can be used to deliver an embolic coil from a delivery device. In certain embodiments, the saline flush can be used in conjunction with a pusher wire.

As another example, in some embodiments, multiple (e.g., two, three, four) embolic coils can be delivered using one delivery device.

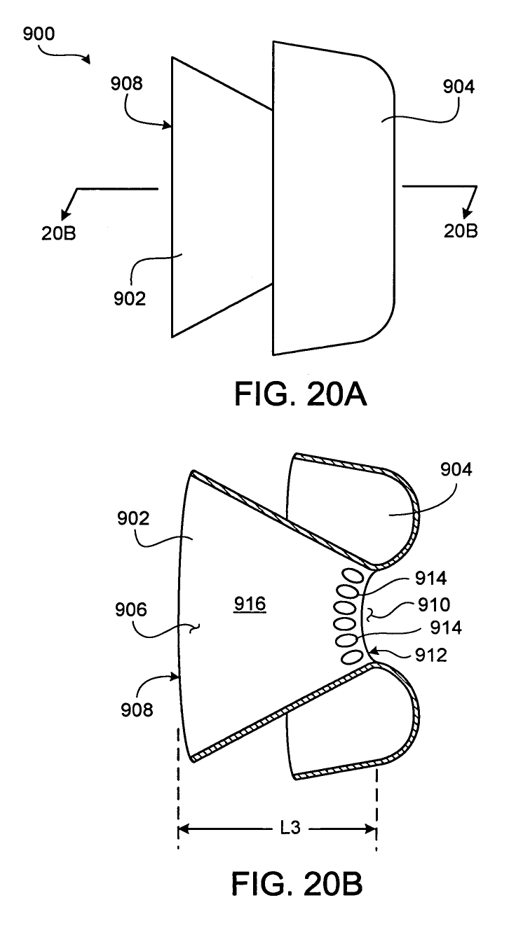

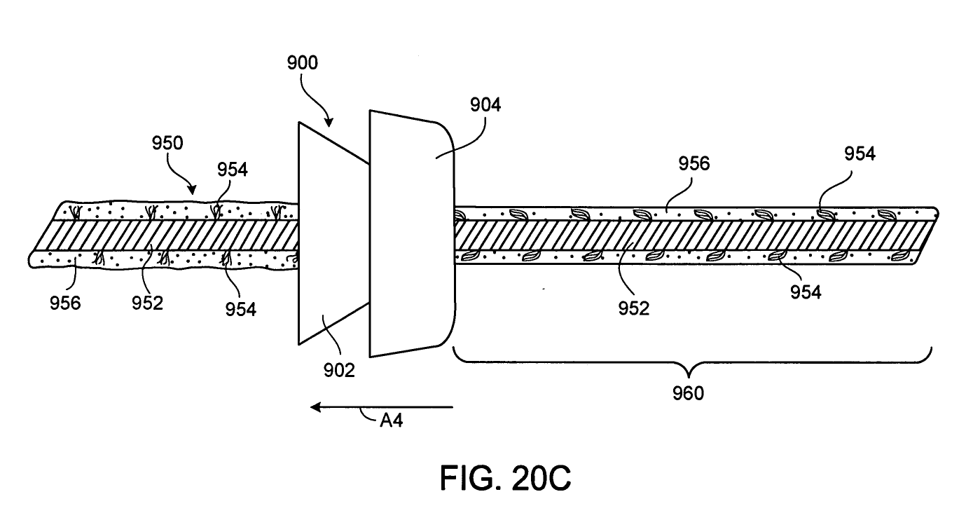

As an additional example, in some embodiments, a device can be used to orient and/or align the fibers of a coated fibered embolic coil, and/or to smoothen the coating on a coated embolic coil. For example, FIGS. 20A and 20B show a device 900 including a cone-shaped region 902 surrounded by a moat-shaped region 904. Cone-shaped region 902 and/or moat-shaped region 904 can be formed of, for example, one or more polymers, such as low molecular weight polypropylene and/or nylon. As shown in FIG. 20B, cone-shaped region 902 has a length L3 that can be, for example, about one inch. Cone-shaped region 902 has a relatively large hole 906 at one end 908, and a smaller hole 910 at another end 912. Cone-shaped region 902 also includes multiple apertures 914 in its wall 916, near end 912. In some embodiments, the minimum distance between an aperture 914 and end 912 of cone-shaped region 902 can be at least 0.001 inch and/or at most about 0.25 inch (e.g., 0.125 inch).

FIG. 20C shows the use of device 900 on a coated fibered embolic coil 950. As shown in FIG. 20C, device 900 is disposed around coil 950. Coil 950 extends through both hole 906 and hole 910 of cone-shaped region 902. Coil 950 includes a coil body 952, fibers 954, and a coating 956 over coil body 952 and fibers 954. Device 900 is pulled over coil 950 in the direction of arrow A4, causing coating 956 to become smoother, and also aligning and orienting fibers 954 so that fibers 954 are closer to coil body 952. During this process, excess coating material from coating 956 travels through apertures 914 of cone-shaped region 902, and into moat-shaped region 904. Moat-shaped region 904 traps the excess coating material and limits the likelihood that the excess coating material will fall back onto the smoothened region 960 of coil 950.

By bringing fibers 954 closer to coil body 952, device 900 can cause the overall profile of coil 950 to decrease. As the profile of coil 950 decreases, the deliverability of coil 950 can increase (e.g., because coil 950 can be relatively unlikely to become stuck within a delivery device).

In certain embodiments, by bringing fibers 954 closer to coil body 952, device 900 can cause the effective column strength (and, therefore, the pushability) of coil 950 to increase. This process can laminate the fibers to the coil, forming a robust & stream lined overall coil profile.

As a further example, in certain embodiments, a treatment site can be occluded by using embolic coils in conjunction with other occlusive devices. For example, embolic coils can be used with embolic particles, such as those described in Buiser et al., U.S. Patent Application Publication No. US 2003/0185896 A1, published on Oct. 2, 2003, and entitled “Embolization”, and in Lanphere et al., U.S. Patent Application Publication No. US 2004/0096662 A1, published on May 20, 2004, and entitled “Embolization”, both of which are incorporated herein by reference. In some embodiments, embolic coils can be used in conjunction with one or more embolic gels. Embolic gels are described, for example, in Richard et al., U.S. Patent Application Publication No. US 2006/0045900 A1, published on Mar. 2, 2006, and entitled “Embolization”, which is incorporated herein by reference.

As an additional example, in some embodiments, an embolic coil can include one or more radiopaque markers. The radiopaque markers can, for example, be attached to one or more windings of the embolic coil.

As a further example, in some embodiments, a wire that is used to form an embolic coil can be coated. A wire can be coated using, for example, one or more of the methods described above with reference to coating a coil body.

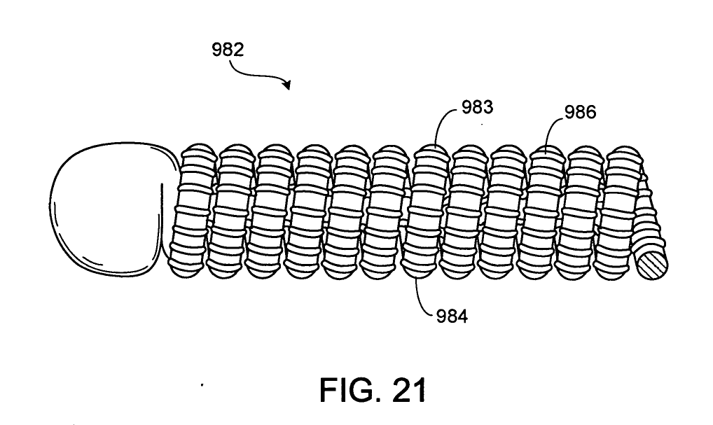

As another example, in certain embodiments, a coil body and/or wire can be coated by forming a sheath of a coating material (e.g., polytetrafluoroethylene (PTFE)) and placing the sheath around the coil body and/or wire. In some embodiments, the sheath can be shrunk (e.g., heat-shrunk) around the coil body and/or wire. In certain embodiments, a coil body and/or wire can be coated by wrapping one or more fibers (e.g., thermally extruded fibers), wires, and/or ribbons of a coating material around the coil body and/or wire. For example, FIG. 21 shows an embolic coil 982 including a coil body 983 formed of windings of a wire 984. Polymeric fibers 986 are wrapped around wire 984. Embolic coils including fibers are described, for example, in Wallace et al., U.S. Pat. No. 6,280,457, which is incorporated herein by reference.

As an additional example, in some embodiments, an introducer sheath can include different regions with different outer diameters. For example, in certain embodiments, an introducer sheath can include a region (e.g., a proximal region) having an outer diameter of from 0.04 inch to 0.1 inch, and a region (e.g., a distal region) having an outer diameter of from 0.02 inch to 0.035 inch. In some embodiments, an introducer sheath can have a tapered outer diameter. In certain embodiments, the outer diameter of a distal region of an introducer sheath can be selected to mate with a hub of a particular microcatheter.

Other embodiments are in the claims.

What is claimed is:

1. An article, comprising:
an embolic coil body;
at least one fiber attached to the embolic coil body; and
a first material supported by at least one member selected from the group consisting of the embolic coil body and the at least one fiber.

2. The article of claim 1, wherein the first material is bioerodible.

3. The article of claim 2, wherein the first material is bioabsorbable.

4. The article of claim 1, wherein the first material is bioabsorbable.

5. The article of claim 1, wherein the first material is supported by the embolic coil body.

6. The article of claim 5, wherein the first material is supported by the at least one fiber.

7. The article of claim 5, wherein the first material is not supported by the at least one fiber.

8. The article of claim 1, wherein the first material is supported by the at least one fiber.

9. The article of claim 8, wherein the first material is not supported by the embolic coil body.

10. The article of claim 1, wherein the embolic coil body comprises a plurality of windings of at least one wire.

11. The article of claim 10, wherein the first material is supported by the at least one wire.

12. The article of claim 11, wherein the at least one wire comprises a metal or a metal alloy.

13. The article of claim 1, wherein the first material is in the form of a coating on the embolic coil body.

14. The article of claim 1, wherein the first material is in the form of a coating on the at least one fiber.

15. The article of claim 1, wherein the first material comprises a gel.

16. The article of claim 1, further comprising a therapeutic agent.

17. The article of claim 16, wherein the therapeutic agent is dispersed within the first material.

18. The article of claim 1, wherein the first material comprises a cellulose ester, a polyurethane, a methacrylate, polyvinylpyrrolidone, or a combination thereof.

19. The article of claim 1, wherein the first material comprises a carbohydrate.

20. The article of claim 1, wherein the first material comprises a polymer.

21. The article of claim 20, further comprising a second material.

22. The article of claim 21, wherein the second material is combined with the first material.

23. The article of claim 21, wherein the second material is dispersed within the first material.

24. The article of claim 21, wherein the second material comprises a nitric oxide donor.

25. The article of claim 21, wherein the second material is bioerodible.

26. The article of claim 25, wherein the second material is bioabsorbable.

27. The article of claim 21, wherein the second material is bioabsorbable.

28. The article of claim 21, wherein the second material comprises a polymer.

29. An article, comprising:
an embolic coil body;
a plurality of fibers attached to the embolic coil body; and
a coating comprising a gel,
wherein the coating contacts the embolic coil body and the plurality of fibers.

30. A medical device, comprising:
a tubular body defining a lumen; and
at least one article disposed within the lumen, the at least one article comprising:
an embolic coil body;
at least one fiber attached to the embolic coil body; and
a material supported by at least one member selected from the group consisting of the embolic coil body and the at least one fiber.

31. The medical device of claim 30, wherein the tubular body comprises a catheter.

32. The medical device of claim 30, wherein the tubular body comprises an introducer sheath.

33. A method, comprising:
administering an article to a subject,
wherein the article comprises:
an embolic coil body;
at least one fiber attached to the embolic coil body; and
a material supported by at least one member selected from the group consisting of the embolic coil body and the at least one fiber.

34. A method, comprising:
administering a medical device to a subject,
wherein the medical device comprises:
a tubular body defining a lumen; and
at least one article disposed within the lumen, the at least one article comprising:
an embolic coil body;
at least one fiber attached to the embolic coil body; and
a material supported by at least one member selected from the group consisting of the embolic coil body and the at least one fiber.
